(19) United States
(12) Patent Application Publication
(10) Pub. No.: US 2013/0274180 A1
(43) Pub. Date: Oct. 17, 2013

(54) Pharmaceutical Compositions

(71) Applicant: Vertex Pharmaceuticals Incorporated, (US)

(72) Inventors: Yong Cui, Mountain View, CA (US); Maura Murphy, Baltimore, MD (US); Kirk Dinehart, Holliston, MA (US); Patricia Hurter, Harvard, MA (US); Patrick R. Connelly, Harvard, MA (US); Riccardo N. Panicucci, Billerica, MA (US)

(21) Appl. No.: 13/861,791

(22) Filed: Apr. 12, 2013

Related U.S. Application Data
(63) Continuation of application No. 11/147,524, filed on Jun. 8, 2005, now abandoned.

(51) Int. Cl. A61K 38/07

(52) U.S. Cl. CPC A61K 38/07
USPC 514/4.3

(57) ABSTRACT

Forms and formulations of VX-950 and uses thereof.

### CLAIM OF PRIORITY

This application claims priority under 35 USC §119(e) to U.S. Patent Application Ser. No. 60/578,043, filed on Jun. 8, 2004, the entire contents of which are hereby incorporated by reference.

### TECHNICAL FIELD

This invention relates to pharmaceutical compositions.

### BACKGROUND

Infection by hepatitis C virus (“HCV”) is a compelling human medical problem. HCV is recognized as the causative agent for most cases of non-A, non-B hepatitis, with an estimated human sero-prevalence of 3% globally [A. Alberti et al., “Natural History of Hepatitis C,” J. Hepatology, 31, (Suppl. 1), pp. 17-24 (1999)]. Nearly four million individuals may be infected in the United States alone [M. J. Alter et al., “The Epidemiology of Viral Hepatitis in the United States, Gastroenterol. Clin. North Am., 23, pp. 437-455 (1994); M. J. Alter “Hepatitis C Virus Infection in the United States,” J. Hepatology, 31, (Suppl. 1), pp. 88-91 (1999)].

Upon first exposure to HCV only about 20% of infected individuals develop acute clinical hepatitis while others appear to resolve the infection spontaneously. In almost 70% of instances, however, the virus establishes a chronic infection that persists for decades [S. Iwarson, “The Natural Course of Chronic Hepatitis,” FEMS Microbiology Reviews, 14, pp. 201-204 (1994); D. Lavanchy, “Global Surveillance and Control of Hepatitis C,” J. Viral Hepatitis, 6, pp. 35-47 (1999)]. This usually results in recurrent and progressively worsening liver inflammation, which often leads to more severe disease states such as cirrhosis and hepatocellular carcinoma [M. C. Kew, “Hepatitis C and Hepatocellular Carcinoma”, FEMS Microbiology Reviews, 14, pp. 211-220 (1994); I. Saito et. al., “Hepatitis C Virus Infection is Associated with the Development of Hepatocellular Carcinoma,” Proc. Natl. Acad. Sci. USA, 87, pp. 6547-6549 (1990)]. It is estimated that HCV infects 170 million persons worldwide. Over the next ten years, as a larger proportion of patients who are currently infected enter the third decade of their infection, the number of deaths attributed to hepatitis C is expected to significantly increase. Unfortunately, there are no broadly effective treatments for the debilitating progression of chronic HCV.

There are not currently any fully satisfactory anti-HCV agents or treatments. Interferon is used to treat HCV, as well as pegylated Interferon, which can also be dosed in combination with Ribavirin. Any treatment regimen containing Interferon is known to have significant side effects, and a there is thus a significant unment medical need for a safe, effective, oral therapy to treat Hepatitis C virus. Moreover, the prospects for effective anti-HCV vaccines remain uncertain.

VX-950 is a competitive, reversible peptidomimetic HCV NS3/4A protease inhibitor with a steady state binding constant (ki*) of 3 nM (and with a Ki of 8 nM) [WO 02/018369].

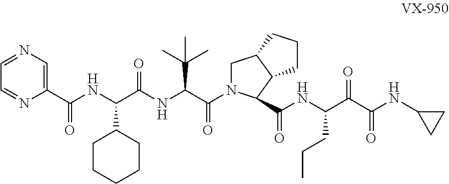 [SMILES: CCC[C@H](NC(=O)[C@@H]1[C@H]2CCC[C@H]2CN1C(=O)[C@@H](NC(=O)[C@@H](NC(=O)c1cnccn1)C1CCCCC1)C(C)(C)C)C(=O)C(=O)NC1CC1]

VX-950 is highly insoluble in water.

### SUMMARY

The inventors have discovered fauns and formulations of VX-950 having improved bioavailability relative to crystalline VX-950. These forms and formulations are useful for treating HCV infection.

Accordingly, in one aspect, the invention features a preparation of amorphous VX-950, for example a preparation of VX-950 that is substantially pure of impurities and/or crystalline VX-950. For example, in one embodiment, the invention features formulations containing VX-950 in the amorphous form, which enhances the metastable solubility of VX-950 relative to the crystalline form, and thus provides improved bioavailability. The invention includes a number of possible formulations, all of which contain VX-950 in the amorphous form.

In one aspect, the invention features a composition including amorphous VX-950 and a second component. The second component can be selected from a variety of components, including, for example, a surfactant, polymer, or inert pharmaceutically acceptable substance. In some preferred embodiments, the composition comprises a solid dispersion, a mixture or a liquid dispersion. In some embodiments, the composition is in the form of a solid (e.g., a tablet or capsule).

In another aspect, the invention features a solid dispersion of amorphous VX-950.

In some embodiments, the solid dispersion includes less than about 40% of crystalline VX-950 (e.g., less than about 35%, less than about 30%, less than about 20%, less than about 10%, less than about 5%, or less than about 1%). For example, in some embodiments, the solid dispersion is substantially free of crystalline VX-950.

In some embodiments, the solid dispersion further includes a surfactant, polymer, or inert pharmaceutically acceptable substance. For example, the solid dispersion can include a polymer, and the polymer can include one or more than one water-soluble polymer or partially water-soluble polymer.

In some embodiments, the VX-950 has improved physical or chemical stability relative to amorphous VX-950 without the presence of polymer. In some embodiments the solid dispersion has a higher glass transition temperature than the glass transition temperature of neat amorphous VX-950. In some embodiments, the VX-950 has a relaxation rate that is lower than the relaxation rate of neat amorphous VX-950.

In some embodiments, the solid dispersion includes a polymer that is present in sufficient amount such that following an administration of the solid dispersion, the level of VX-950 in the blood of a rat is at least about 20% higher than seen with an administration of VX-950 which does not include a polymer, for example, at least about 50% higher, at least about 100% higher, at least about 200% higher, at least about 300% higher or at least about 400% higher.

In some embodiments, the solid dispersion includes a cellulosic polymer, for example an HPMC polymer or an HPMCAS polymer.

In some embodiments, the polymer is present in the solid dispersion in an amount of from about 10% by weight to about 80%, for example from about 30% to about 75%, for example, about 70%, about 50%, or about 49.5% by weight.

In some embodiments, VX-950 is present in the solid dispersion in an amount of from about 10% by weight to about 80% by weight, for example from about 30% to about 75%, for example, about 70%, about 50%, or about 49.5% by weight. In some embodiments, VX-950 is present in the solid dispersion in an amount of greater than about 80%.

In some embodiments the solid dispersion includes a surfactant, for example sodium lauryl sulfate or Vitamin E TPGS.

The amount of surfactant present in the solid dispersion is dependent on a variety of factors, including, for example, the chemical nature of the surfactant. In some embodiments, the surfactant is present in an amount from about 0.1 to about 15%, for example from about 0.1% to about 5%, preferably about 1%.

In some embodiments, substantially all of the VX-950 is present in the solid dispersion in amorphous form.

In some embodiments, the VX-950 is a mixture of the L-isomer and the D-isomer.

In some embodiments, the VX-950 is substantially pure L-isomer.

In some embodiments, the solid dispersion is obtained by spray drying.

In one embodiment, the invention provides a solid dispersion of VX-950, such as an amorphous solid dispersion. For example an amorphous solid dispersion including VX-950, at least one polymer, and optionally one or more solubility enhancing surfactant is provided. The dispersion can enhance the aqueous solubility and bioavailability of VX-950 upon oral dosing of the solid dispersion to a mammal (e.g., a rat, dog or human). In certain aspects, at least a portion of the VX-950 in the solid dispersion is in the amorphous state (e.g., at least about 50%, at least about 55%, at least about 60%, at least about 65%, at least about 70%, at least about 75%, at least about 80%, at least about 85%, at least about 90%, at least about 95%, at least about 98%, or at least about 99%). In preferred embodiments, the solid dispersion is essentially or substantially free of crystalline VX-950.

In certain solid dispersions, VX-950 (e.g., amorphous VX-950) is present in an amount of up to about 99%, for example up to about 98%, up to about 95%, up to about 90%, up to about 85%, up to about 80%, up to about 70%, preferably up to about 70%, up to about 65%, up to about 60%, up to about 55%, and more preferably up to about 50% of the total weight of the solid dispersions. In other embodiments, VX-950 is present in an amount of at least about 1% of the solid dispersion, for example at least about 2%, at least about 3%, at least about 4%, preferably at least about 5%, at least about 6%, at least about 7%, at least about 8%, at least about 9%, more preferably at least about 10%, and even more preferably at least about 50%. As shown in the examples herein, a solid dispersion, wherein the VX-950 is present in an amount of about 50% by weight (and more specifically about 49.5%) is included within this invention.

In some embodiments, when VX-950 is in a solid dispersion, at least about 60% by weight of the VX-950 is in an amorphous form, for example, at least about 65%, at least about 70%, at least about 75%, preferably at least about 80%, at least about 85%, at least about 90%, at least about 95%, at least about 98%, or at least about 99%. Dispersions wherein all or substantially all the VX-950 is in amorphous form, are also included.

In some embodiments, a dispersion including VX-950 includes a mixture of the L-isomer and the D-isomer (e.g., 1:1) of VX-950, or VX-950 may be in a substantially pure form of either isomer. For example, mixtures of about 60:40 of L:D (+/−5%) are included. In certain embodiments, the VX-950 is in an amount of about 95%, about 98%, or greater than about 98% of the L-isomer.

An amorphous solid dispersion generally exhibits a glass transition temperature, where the dispersion makes a transition from a glassy solid to a rubbery composition. In general, the higher the glass transition temperature, the greater the physical stability of the dispersion. The existence of a glass transition temperature generally indicates that at least a large portion of the composition (e.g., dispersion) is in an amorphous state. The glass transition temperature (T_{g}) of a solid dispersion suitable for pharmaceutical applications is generally at least about 50° C. In some embodiments, higher temperatures are preferred. Therefore, in some embodiments, a solid dispersion of this invention has a T_{g} of at least about 100° C. (e.g., at least about 100° C., at least about 105° C., at least about 110° C., at least about 115° C., at least about 120° C., at least about 125° C., at least about 130° C., at least about 135° C., at least about 140° C., at least about 150° C., at least about 160° C., at least about 170° C., at least about 175° C., at least about 180° C., or at least about 190° C.). In some preferred embodiments, the T_{g} is up to about 200° C. Unless otherwise noted, the glass transition temperatures described herein are measured under dry conditions.

In another aspect, the invention provides amorphous VX-950. Amorphous VX-950, without the addition or presence of any polymers or other excipients, enhances the aqueous solubility and the bioavailability of VX-950 (as compared to crystalline VX-950) upon oral dosing to mammals.

In another aspect, the invention features pharmaceutical compositions of amorphous VX-950. In some embodiments, the amorphous VX-950 is substantially free of crystalline VX-950.

In another aspect, the invention features a pharmaceutical composition including an amorphous VX-950 as a solid dispersion and one or more of a surfactant, polymer, inert pharmaceutically acceptable substance, or pharmaceutically acceptable carrier.

In some embodiments, the composition includes a polymer and the polymer is one or more than one water-soluble polymer or partially water-soluble polymer.

In some embodiments, the VX-950 has improved physical or chemical stability relative to crystalline VX-950. In some embodiments, the solid dispersion has a higher glass transition temperature than the glass transition temperature of neat amorphous VX-950. In some embodiments, the VX-950 has a relaxation rate that is lower than the relaxation rate of neat amorphous VX-950.

In some embodiments, the pharmaceutical composition includes a polymer in a sufficient amount such that following an administration of the solid dispersion, the level of VX-950 in the blood of a rat is at least 20% higher than seen with an administration of VX-950 which does not include a polymer, for example at least 50%, at least 100%, at least 200%, at least 300% or at least 400% higher.

In some embodiments the polymer is a cellulosic polymer such as HPMC or HPMCAS.

In some embodiments, the invention features pharmaceutical composition including: an amorphous solid dispersion of VX-950 wherein said VX-950 comprises 30-75% wt/wt of the pharmaceutical composition, one or more polymer selected from the group of HPMC and HPMCAS, wherein said polymer is comprises 30-75% wt/wt of the pharmaceutical composition, and a surfactant, wherein said surfactant comprises 0.5-2% wt/wt of the pharmaceutical composition. As described, the weight percentage of components is relative to the weight of the solid dispersion, which can be further formulated, for example, into a liquid suspension or a tablet.

In some embodiments, the polymer is HPMC or HPMCAS.

In some embodiments, the surfactant is sodium laurel sulfate or Vitamin E TPGS.

In some embodiments, the pharmaceutical composition includes the following components, wherein: said VX-950 includes about 49.5% wt/wt of the pharmaceutical composition, said polymer is HPMC and includes about 49.5% wt/wt of the pharmaceutical composition, and a said surfactant is sodium laurel sulfate or Vitamin E TPGS and includes about 1% wt/wt of the pharmaceutical composition. As described, the weight percentage of components is relative to the weight of the solid dispersion, which can be further formulated, for example, into a liquid suspension or a tablet.

In some embodiments, the pharmaceutical composition includes the following components, wherein: said VX-950 includes about 49.5% wt/wt of the pharmaceutical composition, said polymer is HPMCAS and includes about 49.5% wt/wt of the pharmaceutical composition, and a said surfactant is sodium laurel sulfate or Vitamin E TPGS and includes about 1% wt/wt of the pharmaceutical composition. As described, the weight percentage of components is relative to the weight of the solid dispersion, which can be further formulated, for example, into a liquid suspension or a tablet.

In some embodiments, the pharmaceutical composition includes the following components, wherein said VX-950 includes about 70% wt/wt of the pharmaceutical composition, said polymer is HPMC or HPMCAS and includes about 29% wt/wt of the pharmaceutical composition, and said surfactant is sodium laurel sulfate of Vitamin E TPGS and includes about 1% wt/wt of the pharmaceutical composition. As described, the weight percentage of components is relative to the weight of the solid dispersion, which can be further formulated, for example, into a liquid suspension or a tablet.

In another aspect, the invention features a pharmaceutical composition including; an aqueous suspension comprising amorphous VX-950 particles and a polymer in solution selected from the group of HPMC and HPMCAS.

In some embodiments, the amorphous VX-950 is in the form of a solid dispersion.

In some embodiments, the pharmaceutical composition also includes a surfactant, either in the solution or as a component of the VX-950 particles or both. The surfactant can be, for example, SLS or Vitamin E TPGS.

In some embodiments, the polymer is either in the solution or as a component of the VX-950 particles or both.

In some embodiments, the aqueous suspension includes from about 0.1% to about 20% by weight of the surfactant. In some embodiments, the aqueous suspension includes from about 1 mg/ml to about 100 mg/ml by weight of amorphous VX-950. In some embodiments, the aqueous suspension includes from about 0.1% to about 2.0% by weight of polymer, for example about 1% by weight of polymer.

In some embodiments, the invention includes methods of preparing a form, dispersion, composition, or formulation described herein.

Accordingly, a process for preparing an amorphous form of VX-950 including spray-drying is described. One embodiment provides a process preparing an amorphous form of VX-950 by combining VX-950 and a suitable solvent to form a mixture and then spray-drying the mixture to obtain the amorphous form of VX-950. The mixture may be either a solution or a suspension.

In another aspect, the invention features a process for preparing an amorphous form of VX-950 including spray-drying VX-950 to provide an amorphous form of VX-950.

In some embodiments, the process includes combining VX-950 and a suitable solvent to form a mixture and then spray-drying the mixture to obtain the amorphous form of VX-950.

In some embodiments, the process includes

a) forming a mixture VX-950, a polymer, and a solvent; and

b) spray-drying the mixture to form a solid dispersion comprising VX-950.

In some embodiments, the polymer is HPMC or HPMCAS.

In some embodiments, the polymer is present in an amount of from about 30% to about 70% by weight in the solid dispersion.

In some embodiments, the mixture also includes a surfactant, for example, sodium lauryl sulfate (SLS) or Vitamin E TPGS.

In some embodiments, the solvent includes methylene chloride. In some embodiments, the solvent includes acetone. In some embodiments, the solvent includes a mixture of methylene chloride and acetone. For example, the solvent can include from about 0% to about 30% acetone and from about 70% to about 100% methylene chloride, or the solvent can includes from about 0% to about 40% acetone and from about 60% to about 100% methylene chloride. Other exemplary ratios of methylene chloride to acetone include 80:20, 75:25, and 70:30.

In another aspect, the invention features a solid dispersion prepared according to a process described herein.

This invention also provides a process for preparing a solid dispersion of VX-950 comprising:

a) forming a solution of VX-950, a polymer (e.g., crystallization inhibiting or a stabilizing polymer), and a solvent;

b) rapidly removing the solvent from the solution to form a solid amorphous dispersion comprising VX-950 and the crystallization inhibiting polymer. In certain embodiments, the solvent is removed by spray drying.

As would be appreciated spray drying may be done in the presence of an inert gas. In certain embodiments, processes that involve spray drying may be done in the presence of a supercritical fluid involving carbon dioxide or a mixture of carbon dioxide.

Accordingly, in another embodiment, this invention provides a process for preparing a solid dispersion of VX-950 comprising

a) forming a mixture of VX-950, a polymer (e.g., a supporting polymer, a crystallization inhibiting polymer, or stabilizing polymer), and a solvent; and

b) spray-drying the mixture to form a solid dispersion comprising VX-950.

These processes could be used to prepare the compositions of this invention. The amounts and the features of the components used in the processes would be as described herein.

In another aspect, the invention features a method of treating HCV infection in a mammal. In one embodiment, the method includes administering amorphous VX-950, wherein the amorphous VX-950 is as defined herein. In another embodiment, the method includes administering a solid dispersion described herein.

In another embodiment, the method includes administering an additional agent selected from an immunomodulatory agent; an antiviral agent; another inhibitor of HCV NS3/4A protease; another inhibitor of IMPDH; an inhibitor of a target in the HCV life cycle other than NS3/4A protease; an inhibitor of internal ribosome entry, a broad-spectrum viral inhibitor; a cytochrome P-450 inhibitor; or combinations thereof.

In another aspect, the invention features pharmaceutical packs or kits including a VX-950 composition described herein or amorphous VX-950.

An amorphous form of a drug may exhibit different properties than the crystalline form (see, U.S. Pat. No. 6,627,760). Embodiments of the invention include amorphous VX-950, which thermodynamically is at a higher energy level than its corresponding crystalline form. Therefore, it is energetically more active, and thus often exhibits higher metastable solubility, faster dissolution behavior, as well as less stable physical properties. The first two properties act to enhance the aqueous solubility and bioavailability of the drug, while the last may be detrimental to this goal by presenting a physically less stable composition, of which the bioavailability may change due to recrystallization of the drug from its amorphous state during storage, or upon administration to humans or animals.

To improve the stability of an amorphous solid (which is generally less stable than a crystal form), a polymer or polymeric mixture can be used to form an amorphous solid dispersion system together with the drug. In some embodiments, a “solid solution”, which is a system which will not phase separate over time, or a solid dispersion can be formulated in which the recrystallization of the drug is effectively inhibited during a pharmaceutically significantly long period (e.g., two years) at ambient temperature.

In preferred embodiments, release of a polymer from a solid dispersion, where the solid dispersion contains both VX-950 and the polymer, into an aqueous solution can reduce solution mediated crystallization of the VX-950 which is solubilized in the aqueous media after being released from the solid dispersion. For example, when a solid dispersion of VX-950 is introduced into an aqueous biological fluid, for example as is found in the stomach or small intestine, co-release or advanced-release of polymer, for example, HMPC or HPMCAS, with amorphous VX-950 will reduce crystallization of VX-950 in the aqueous biological fluid, thereby enhancing one or more of bioavailability, solubility and absorption of VX-950. Furthermore, inclusion of such a polymer, either in the aqueous medium or in combination with VX-950, can reduce crystallization of VX-950 in the aqueous medium in vitro, e.g., in the preparation of liquid formulations of VX-950.

The manufacture of an amorphous solid dispersion containing VX-950 presented several challenges. First, VX-950 does not dissolve to a significant amount in water or most other conventional organic solvents, including acetone, ethyl acetate, and acetonitrile. The aqueous solubility of VX-950 at room temperature is virtually undetectable by HPLC and the aqueous solubility is not pH-dependent. Second, VX-950 has shown chemical reactivity with some alcohols, for example, MeOH, EtOH, and iPrOH, which makes these unsuitable solvents. Third, the melting point of VX-950 is about 240° C., making hot-melt technologies somewhat impractical due to the potential degradation of VX-950 at the high temperature. Therefore, an appropriate solvent or solvent mixture is crucial to optimizing the processing and production of a solid dispersion.

Amorphous solid dispersions of the invention can significantly improve the oral bioavailability of VX-950. In the presence of an appropriate surfactant or surfactant mixture (e.g., SLS or Vitamin E d-alpha tocopheryl polyethylene glycol 1000 succinate (Vitamin E TPGS)), the bioavailability can be further enhanced.

Amorphous solid dispersions of the invention can provide improved bioavailability of VX-950 when orally administered relative to the administration of crystalline VX-950. In some embodiments, these solid dispersions are in a solid state that can be conveniently stored and administered. The manufacture of the solid dispersions can be conducted and scaled up successfully by selecting an organic solvent or solvent mixture (for example, methylene chloride, acetone, etc.) or a supercritical fluid (for example, involving carbon dioxide). In some embodiments, solid dispersions can have improved chemical and physical stability. For example, in some instances the solid dispersions can be chemically and/or physically stable for at least two years at conventional storage conditions (room temperature).

The details of one or more embodiments of the invention are set forth in the accompanying description below. Other features, objects, and advantages of the invention will be apparent from the description and from the claims.

### DESCRIPTION OF DRAWINGS

FIG. 1 depicts a comparison between various compositions VX-950.

FIG. 2 depicts a comparison of rat pK between various compositions that include VX-950.

FIGS. 3-6 depict comparisons of stability data for various suspensions including VX-950 and Vitamin E TPGS.

FIGS. 7-10 depict comparisons of kinetic solubility data for various suspensions including VX-950 and Vitamin E TPGS.

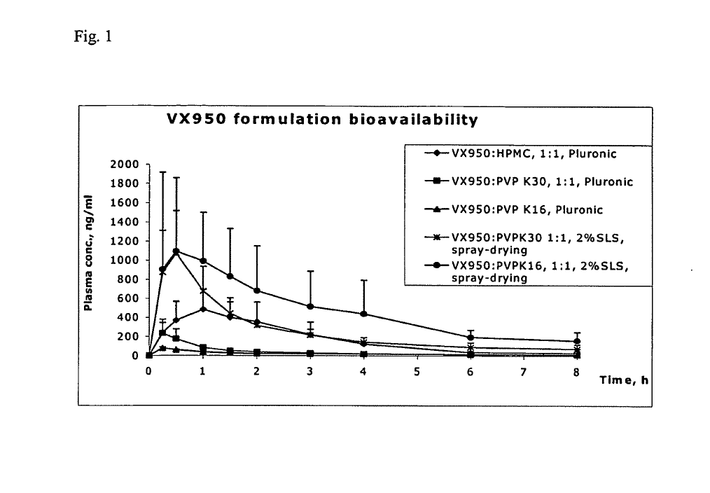

### DETAILED DESCRIPTION

In general, it has been found that absolute bioavailability after orally administering a micronized crystalline drug powder of VX-950 to rats is less than 0.5%. Simple mixtures of VX-950 with conventional pharmaceutical excipients exhibit similarly low bioavailability upon oral administration to mammals. Compositions including VX-950 in crystalline form (i.e., where a significant portion of VX-950 is in crystalline form) generally do not achieve drug absorption to an extent that provides for sufficient therapeutic effects of VX-950. The compositions described herein provide comparatively improved bioavailability. Accordingly, in some embodiments, a preparation of amorphous VX-950 is provided. For example a purified preparation that is substantially free of impurities, including crystalline VX-950. In some embodiments the invention includes a pharmaceutical composition in the form of a solid dispersion comprising VX-950. The compositions of this invention are stable, easy to administer, and give high bioavailability of VX-950 upon administration.

In certain embodiments, the VX-950 is present in an amount of from about 5% to about 90% by weight, for example from about 5% to about 70%, preferably up to about 50% by weight. The VX-950 is a mixture of the D-isomer and L-isomer or is a substantially pure product of either isomer. The VX-950 is preferably substantially amorphous (e.g., at least about 50% of VX-950 is amorphous, at least about 55% of VX-950 is amorphous, at least about 60% of VX-950 is amorphous, at least about 65% of VX-950 is amorphous, at least about 70% of VX-950 is amorphous, at least about 75% of VX-950 is amorphous, at least about 80% of VX-950 is amorphous, at least about 85% of VX-950 is amorphous, at least about 90% of VX-950 is amorphous, at least about 95% of VX-950 is amorphous, at least about 98% of VX-950 is amorphous, at least about 99% of VX-950 is amorphous, or substantially all of VX-950 is amorphous.

As used herein, the term “amorphous” refers to a solid material having no long range order in the position of its atoms. Amorphous solids are generally supercooled liquids in which the molecules are arranged in a random manner so that there is no well-defined arrangement and no long range order. Amorphous solids are generally isotropic, i.e. exhibit similar properties in all directions and do not have definite melting points. For example, an amorphous material is a solid material having no sharp characteristic crystalline peak(s) in its X-ray power diffraction (XRPD) pattern (i.e., is not crystalline as determined by XRPD). Instead, one or several broad peaks (e.g., halos) appear in its XRPD pattern. Broad peaks are characteristic of an amorphous solid. See, US 2004/0006237 for a comparison of XRPDs of an amorphous material and crystalline material.

As used herein “crystalline solids” refers to compounds or compositions where the structural units are arranged in fixed geometric patterns or lattices, so that crystalline solids have rigid long range order. The units that constitute the crystal structure can be atoms, molecules, or ions. Crystalline solids show definite melting points.

As used herein, a “dispersion” refers to a disperse system in which one substance, the dispersed phase, is distributed, in discrete units, throughout a second substance (the continuous phase or vehicle). The size of the dispersed phase can vary considerably (e.g. colloidal particles of nanometer dimension, to multiple microns in size). In general, the dispersed phases can be solids, liquids, or gases. In the case of a solid dispersion, the dispersed and continuous phases are both solids. In pharmaceutical applications, a solid dispersion can include a crystalline drug (dispersed phase) in an amorphous polymer (continuous phase), or alternatively, an amorphous drug (dispersed phase) in an amorphous polymer (continuous phase). In some embodiments an amorphous solid dispersion includes the polymer constituting the dispersed phase, and the drug constitute the continuous phase.

The term “amorphous solid dispersion” generally refers to a solid dispersion of two or more components, usually a drug and polymer, but possibly containing other components such as surfactants or other pharmaceutical excipients, where the drug is in the amorphous phase, and the physical stability and/or dissolution and/or solubility of the amorphous drug is enhanced by the other components.

A solid dispersion as provided herein is a particularly favorable embodiment of this invention. Solid dispersions typically include a compound dispersed in an appropriate carrier medium, such as a solid state carrier. In one embodiment, a carrier according to this invention comprises a polymer, preferably, a water-soluble polymer or a partially water-soluble polymer. It would be understood that one or more than one water-soluble polymer could be used in a solid dispersion of this invention.

An exemplary solid dispersion is a co-precipitate or a co-melt of VX-950 with at least one polymer. A “Co-precipitate” is a product after dissolving a drug and a polymer in a solvent or solvent mixture followed by the removal of the solvent or solvent mixture. Sometimes the polymer can be suspended in the solvent or solvent mixture. The solvent or solvent mixture includes organic solvents and supercritical fluids. A “co-melt” is a product after heating a drug and a polymer to melt, optionally in the presence of a solvent or solvent mixture, followed by mixing, removal of at least a portion of the solvent if applicable, and cooling to room temperature at a selected rate. In some cases, the solid dispersions are prepared by adding a solution of a drug and a solid polymer followed by mixing and removal of the solvent. To remove the solvent, vacuum drying, spray drying, tray drying, lyophilization, and other drying procedures may be applied. Applying any of these methods using appropriate processing parameters, according to this invention, would provide VX-950 in an amorphous state in the final solid dispersion product.

Production of Amorphous VX-950

Any method for obtaining amorphous forms and solid dispersions could be used in connection with this invention including, for example, those described in US 2003/0186952 (see the documents cited therein at paragraph 1092) and US 2003/0185891). In general, methods that could be used include those that involve rapid removal of solvent from a mixture or cooling a molten sample. Such methods include, but are not limited to, rotational evaporation, freeze-drying (i.e., lyophilization), vacuum drying, melt congealing, and melt extrusion. However, a preferred embodiment of this invention involves amorphous solid dispersion obtained by spray-drying. Accordingly, in another embodiment, this invention provides drying the product obtained by spray drying to remove the solvent.

Preparations disclosed herein, e.g., a pharmaceutical composition, can be obtained by spray-drying a mixture comprising VX-950, a suitable polymer, and an appropriate solvent. Spray drying is a method that involves atomization of a liquid mixture containing, e.g., a solid and a solvent, and removal of the solvent. Atomization may be done, for example, through a nozzle or on a rotating disk.

Spray drying is a process, that converts a liquid feed to a dried particulate form. Optionally, a secondary drying process such as fluidized bed drying or vacuum drying, may be used to reduce residual solvents to pharmaceutically acceptable levels. Typically, spray-drying involves contacting a highly dispersed liquid suspension or solution, and a sufficient volume of hot air to produce evaporation and drying of the liquid droplets. The preparation to be spray dried can be any solution, coarse suspension, slurry, colloidal dispersion, or paste that may be atomized using the selected spray-drying apparatus. In a standard procedure, the preparation is sprayed into a current of warm filtered air that evaporates the solvent and conveys the dried product to a collector (e.g., a cyclone). The spent air is then exhausted with the solvent, or alternatively the spent air is sent to a condenser to capture and potentially recycle the solvent. Commercially available types of apparatus may be used to conduct the spray-drying. For example, commercial spray dryers are manufactured by Buchi Ltd. and Niro (e.g., the PSD line of spray driers manufactured by Niro) (see, US 2004/0105820; US 2003/0144257).

Spray-drying typically employs solids loads of material from about 5% to about 30%, (i.e., drug plus and excipients) preferably at least about 10%. In some embodiments, loads of less than 10% may result in poor yields and unacceptably long run-times. In general, the upper limit of solids loads is governed by the viscosity of (e.g., the ability to pump) the resulting solution and the solubility of the components in the solution. Generally, the viscosity of the solution can determine the size of the particle in the resulting powder product.

Techniques and methods for spray-drying may be found in Perry's Chemical Engineering Handbook, 6th Ed., R. H. Perry, D. W. Green & J. O. Maloney, eds.), McGraw-Hill book co. (1984); and Marshall “Atomization and Spray-Drying” 50, Chem. Eng. Prog. Monogr. Series 2 (1954). In general, the spray-drying is conducted with an inlet temperature of from about 60° C. to about 200° C., for example, from about 70° C. to about 150° C., preferably from about 80° C. to about 110° C., e.g., about 90° C. The spray-drying is generally conducted with an outlet temperature of from about 40° C. to about 100° C., for example from about 50° C. to about 65° C., e.g., about 56° C. or 58° C.

Removal of the solvent may require a subsequent drying step, such as tray drying, fluid bed drying (e.g., from about room temperature to about 100° C.), vacuum drying, microwave drying, rotary drum drying or biconical vacuum drying (e.g., from about room temperature to about 200° C.).

In some instances, it has been found that PVP K29/32 appears to trap solvent within the solid. There is a direct relationship between bulk density/flow and residual solvent; the higher the bulk density/better flow, the higher the residual solvent. It may be advantageous to optimize the powder flow and bulk density and use secondary drying to remove the residual solvent. In one embodiment of this invention, the solid dispersion is fluid-bed dried. Fluid-bed drying at about 75° C. for about 8 hours has been found effective in certain embodiments to provide optimal effects in certain solid dispersion of VX-950. In other embodiments, e.g. using HPMCAS as the polymer in the solid dispersion, fluid-bed drying at 45° C. for about 4 hours has been effective to provide acceptable levels of residual solvent in the final product.

In preferred processes, the solvent includes a volatile solvent. In some embodiments, the solvent includes a mixture of volatile solvents. Preferable solvents include those that can dissolve both VX-950 and the polymer. Suitable solvents include those described above, for example, methylene chloride, acetone, etc. In more preferred processes the solvent is a mixture of methylene chloride and acetone. Although alcoholic solvents could be used in connection with this invention, alcohols have been found to react with VX-950 to form ketals. Accordingly, a solvent that does not react with VX-950 (particularly to form ketals) is preferred. Such a solvent should not contain an OH group or a similarly reactive moiety. In these processes, therefore, a preferred solvent is other than an alcohol.

Because of the reactivity of VX-950, a preferred polymer for use in connection with this invention is other than a polyethylene glycol (e.g., PEG 8000) (i.e., other than a polymer having free hydroxyl moieties).

The particle size and the temperature drying range may be modified to prepare an optimal solid dispersion. As would be appreciated by skilled practitioners, a small particle size would lead to improved solvent removal. Applicants have found however that smaller particles lead to fluffy particles that do not provide optimal solid dispersions of VX-950 for downstream processing such as tabletting. At higher temperatures, crystallization or chemical degradation of VX-950 may occur. At lower temperatures, a sufficient amount of the solvent may not be removed. The methods herein provide a optimal particle size and an optimal drying temperature.

Polymers

Solid dispersions including VX-950 and a polymer (or solid state carrier) included herein.

In one embodiment, a polymer in the present invention is able to dissolve in aqueous media. The solubility of the polymers may be pH-independent or pH-dependent. The latter include one or more enteric polymers. The term “enteric polymer” refers to a polymer that is preferentially soluble in the less acidic environment of the intestine relative to the more acid environment of the stomach, for example, a polymer that is insoluble in acidic aqueous media but soluble when the pH is above 5-6. An appropriate polymer should be chemically and biologically inert. In order to improve the physical stability of the solid dispersions, the glass transition temperature (T_{g}) of the polymer should be as high as possible. For example, preferred polymers have a glass transition temperature at least equal to or greater than the glass transition temperature of the drug (e.g., VX-950). Other preferred polymers have a glass transition temperature that is within about 10 to about 15° C. of the drug (e.g., VX-950). Examples of suitable glass transition temperatures of the polymers include at least about 90° C., at least about 95° C., at least about 100° C., at least about 105° C., at least about 110° C., at least about 115° C., at least about 120° C., at least about 125° C., at least about 130° C., at least about 135° C., at least about 140° C., at least about 145° C., at least about 150° C., at least about 155° C., at least about 160° C., at least about 165° C., at least about 170° C., or at least about 175° C. (as measured under dry conditions). Without wishing to be bound by theory, it is believed that the underlying mechanism is that a polymer with a higher T_{g} generally has lower molecular mobility at room temperature, which can be a crucial factor in stabilizing the physical stability of the amorphous solid dispersion.

Additionally, the hygroscopicity of the polymers should be as low as possible. For the purpose of comparison in this application, the hygroscopicity of a polymer or composition is characterized at about 60% relative humidity. In some preferred embodiments, the polymer has less than about 10% water absorption, for example less than about 9%, less than about 8%, less than about 7%, less than about 6%, less than about 5%, less than about 4%, less than about 3%, or less than about 2% water absorption. Cellulosic polymers generally have about 3% water absorption whereas PVP generally has about 9% water absorption. The hygroscopicity can also affect the physical stability of the solid dispersions. Generally, moisture adsorbed in the polymers can greatly reduce the T_{g} of the polymers as well as the resulting solid dispersions, which will further reduce the physical stability of the solid dispersions as described above.

In one embodiment, the polymer is one or more water-soluble polymer(s) or partially water-soluble polymer(s). Water-soluble or partially water-soluble polymers include but are not limited to, cellulose derivatives (e.g., hydroxypropylmethylcellulose (HPMC), hydroxypropylcellulose (HPC)) or ethylcellulose; polyvinylpyrrolidones (PVP); polyethylene glycols (PEG); polyvinyl alcohols (PVA); acrylates, such as polymethacrylate (e.g., Eudragit® E); cyclodextrins (e.g., β-cyclodextin) and copolymers and derivatives thereof, including for example PVP-VA (polyvinylpyrollidone-vinyl acetate). In some preferred embodiments, the polymer is hydroxypropylmethylcellulose (HPMC), such as HPMC E50 or HPMCE15. As discussed herein, the polymer is a pH-dependent enteric polymer. Such pH-dependent enteric polymers include, but are not limited to, cellulose derivatives (e.g., cellulose acetate phthalate (CAP)), hydroxypropyl methyl cellulose phthalates (HPMCP), hydroxypropyl methyl cellulose acetate succinate (HPMCAS), carboxymethylcellulose (CMC) or a salt thereof (e.g., a sodium salt such as (CMC-Na)); cellulose acetate trimellitate (CAT), hydroxypropylcellulose acetate phthalate (HPCAP), hydroxypropylmethyl-cellulose acetate phthalate (HPMCAP), and methylcellulose acetate phthalate (MCAP), or polymethacrylates (e.g., Eudragit® S). In some preferred embodiments, the polymer is hydroxypropyl methyl cellulose acetate succinate (HPMCAS).

In yet another embodiment, the polymer is an insoluble cross-linked polymer, for example a polyvinylpyrrolidone (e.g., Crospovidone).

In embodiments where the drug forms a solid dispersion with a polymer, for example VX-950 with an HPMC or HPMCAS polymer, the amount of polymer relative to the total weight of the solid dispersion is typically at least about 20%, and preferably at least about 30%, for example, at least about 35%, at least about 40%, at least about 45%, or about 50% (e.g., 49.5%). The amount is typically about 99% or less, and preferably about 80% or less, for example about 75% or less, about 70% or less, about 65% or less, about 60% or less, or about 55% or less. In one embodiment, the polymer is in an amount of up to about 50% of the total weight of the dispersion (and even more specifically, between about 48% and 52%, such as about 49%, about 49.5%, about 50%, about 50.5%, or about 51%).

In one of the more specific embodiments of this invention, the polymer is polyvinylpyrrolidone (PVP) (e.g., PVP29/32) and is present in an amount of up to about 50% (or more specifically, about 50%). As disclosed herein, a dispersion comprising about 49.5% PVP K29/32 is included within this invention.

In another embodiment, the invention includes a solid dispersion of VX-950 and a cellulosic polymer, for example an HPMC or an HPMCAS polymer. In some preferred embodiments, the drug (i.e., VX-950) is present in an amount of at least about 20% of the dispersion, for example at least about 25%, at least about 30%, at least about 35%, at least about 40%, at least about 45%, at least about 50%, or even greater. In some preferred embodiments, the drug is present in an amount between about 48% and 52%, such as about 49%, about 49.5%, about 50%, about 50.5%, or about 51%. As described above, the polymer is present in an amount of at least about 20%, and preferably at least about 30%, for example, at least about 35%, at least about 40%, at least about 45%, or about 50% (e.g., 49.5%). The amount is typically about 99% or less, and preferably about 80% or less, for example about 75% or less, about 70% or less, about 65% or less, about 60% or less, or about 55% or less. In one embodiment, the polymer is in an amount of up to about 50% of the total weight of the dispersion (and even more specifically, between about 48% and 52%, such as about 49%, about 49.5%, about 50%, about 50.5%, or about 51%). In some preferred embodiments, the drug and polymer are present in roughly equal amounts, for example each of the polymer and the drug make up about half of the percentage weight of the dispersion. In some preferred embodiments, the dispersion further includes other minor ingredients, such as a surfactant (e.g., SLS or Vitamin E TPGS). In some preferred embodiments, the surfactant is present in less than about 10% by weight of the dispersion, for example less than about 9% by weight, less than about 8% by weight, less than about 7% by weight, less than about 6% by weight, less than about 5% by weight, less than about 4% by weight, less than about 3% by weight, less than about 2% by weight, or about 1% by weight.

In embodiments including a polymer, the polymer should be present in an amount effective for stabilizing the solid dispersion. Stabilizing includes inhibiting or preventing, the crystallization of VX-950. Such stabilizing would inhibit the conversion VX-950 from amorphous to crystalline form. For example, the polymer would prevent at least a portion (e.g., about 5%, about 10%, about 15%, about 20%, about 25%, about 30%, about 35%, about 40%, about 45%, about 50%, about 55%, about 60%, about 65%, about 70%, about 75%, or greater) of VX-950 from going from an amorphous to a crystalline form. Stabilization can be measured, for example, by measuring the glass transition temperature of the solid dispersion, measuring the rate of relaxation of the amorphous material, or by measuring the solubility or bioavailability of VX-950.

Suitable polymers for use in combination with VX-950, for example to form a solid dispersion such as an amorphous solid dispersion, should have one or more of the following properties:

1. The glass transition temperature of the polymer should have a temperature of no less than about 10-15° C. lower than the glass transition temperature of VX-950. Preferably, the glass transition temperature of the polymer is greater than the glass transition temperature of VX-950, and in general at least 50° C. higher than the desired storage temperature of the drug product. For example, at least about 100° C., at least about 105° C., at least about 105° C., at least about 110° C., at least about 120° C., at least about 130° C., at least about 140° C., at least about 150° C., at least about 160° C., at least about 160° C., or greater.

2. The polymer should be relatively non-hygroscopic. For example, the polymer should, when stored under standard conditions, absorb less than about 10% water, for example, less than about 9%, less than about 8%, less than about 7%, less than about 6%, or less than about 5%, less than about 4%, or less than about 3% water. Preferably the polymer will, when stored under standard conditions, be substantially free of absorbed water.

3. The polymer should have similar or better solubility in solvents suitable for spray drying processes relative to that of VX-950. In preferred embodiments, the polymer will dissolve in one or more of the same solvents or solvent systems as VX-950. It is preferred that the polymer is soluble in at least one non-hydroxy containing solvent such as methylene chloride, acetone, or a combination thereof.

4. The polymer, when combined with VX-950, for example in a solid dispersion or in a liquid suspension, should increase the solubility of VX-950 in aqueous and physiologically relative media either relative to the solubility of VX-950 in the absence of polymer or relative to the solubility of VX-950 when combined with a reference polymer. For example, the polymer could increase the solubility of amorphous VX-950 by reducing the amount of amorphous VX-950 that converts to crystalline VX-950, either from a solid amorphous dispersion or from a liquid suspension.

5. The polymer should decrease the relaxation rate of the amorphous substance.

6. The polymer should increase the physical and/or chemical stability of VX-950.

7. The polymer should improve the manufacturability of VX-950.

8. The polymer should improve one or more of the handling, administration or storage properties of VX-950.

9. The polymer should not interact unfavorably with other pharmaceutical components, for example excipients.

The suitability of a candidate polymer (or other component) can be tested using the spray drying methods (or other methods) described herein to form an amorphous composition. The candidate composition can be compared in terms of stability, resistance to the formation of crystals, or other properties, and compared to a reference preparation, e.g., a preparation of 49.5% amorphous VX-950, 49.5% HPMC or HPMCAS, and 1% of a surfactant, e.g., SLS or vitamin E TPGS; or crystalline VX-950. E.g., a candidate composition could be tested to determine whether it inhibits the time to onset of solvent mediated crystallization, or the percent conversion at a given time under controlled conditions, by at least 50%, 75%, 100%, or 110% as well as the reference preparation, or a candidate composition could be tested to determine if it has improved bioavailability or solubility relative to crystalline VX-950.

Surfactants

A solid dispersion or other composition may include a surfactant. A surfactant or surfactant mixture would generally decrease the interfacial tension between the solid dispersion and an aqueous medium. An appropriate surfactant or surfactant mixture may also enhance aqueous solubility and bioavailability of VX-950 from a solid dispersion. The surfactants for use in connection with the present invention include, but are not limited to, sorbitan fatty acid esters (e.g., Spans®), polyoxyethylene sorbitan fatty acid esters (e.g., Tweens®), sodium lauryl sulfate (SLS), sodium dodecylbenzene sulfonate (SDBS) dioctyl sodium sulfosuccinate (Docusate), dioxycholic acid sodium salt (DOSS), Sorbitan Monostearate, Sorbitan Tristearate, hexadecyltrimethyl ammonium bromide (HTAB), Sodium N-lauroylsarcosine, Sodium Oleate, Sodium Myristate, Sodium Stearate, Sodium Palmitate, Gelucire 44/14, ethylenediamine tetraacetic acid (EDTA), Vitamin E d-alpha tocopheryl polyethylene glycol 1000 succinate (TPGS), Lecithin, MW 677-692, Glutanic acid monosodium monohydrate, Labrasol, PEG 8 caprylic/capric glycerides, Transcutol, diethylene glycol monoethyl ether, Solutol HS-15, polyethylene glycol/hydroxystearate, Taurocholic Acid, Pluronic F68, Pluronic F108, and Pluronic F127 (or any other polyoxyethylene-polyoxypropylene co-polymers (Pluronics®) or saturated polyglycolized glycerides (Gelucirs®)). Specific example of such surfactants that may be used in connection with this invention include, but are not limited to, Span 65, Span 25, Tween 20, Capryol 90, Pluronic F108, sodium lauryl sulfate (SLS), Vitamin E TPGS, pluronics and copolymers. SLS and Vitamin E TPGS are preferred.

The amount of the surfactant (e.g., SLS or Vitamin E TPGS) relative to the total weight of the solid dispersion may be between 0.1-15%. Preferably, it is from about 1 to about 10%, more preferably from about 1 to about 5%, e.g., about 1%, about 2%, about 3%, about 4%, or about 5%.

In certain embodiments, the amount of the surfactant relative to the total weight of the solid dispersion is at least about 0.1, preferably at least about 0.5%, and more preferably at least about 1% (e.g., about 1%). In these embodiments, the surfactant would be present in an amount of no more than about 15%, and preferably no more than about 12%, about 11%, about 10%, about 9%, about 8%, about 7%, about 6%, about 5%, about 4%, about 3%, about 2% or about 1%. As shown in the examples herein, an embodiment wherein the surfactant is in an amount of about 1% by weight is preferred.

An especially preferred embodiment includes a solid dispersion of VX-950, HPMC, and a surfactant. For example a solid dispersion including 49.5% VX-950, 49.5% of an HPMC polymer, such as HPMC E50, and 1% of a surfactant such as SLS.

Another especially preferred embodiment includes a solid dispersion of VX-950, HPMCAS, and a surfactant. For example a solid dispersion including 49.5% VX-950, 49.5% of an HPMCAS polymer, and 1% of a surfactant such as SLS.

HPMCAS is available in a variety of grades from ShinEtsu, including AS-LF, AS-MF, AS-HF, AS-LG, AS-MG, AS-HG. Each of these grades vary with the percent substitution of acetate and succinate.

Candidate surfactants (or other components) can be tested for suitability for use in the invention in a manner similar to that described for testing polymers.

Compositions/Dosage/Packaging/Use

Pharmaceutical compositions are also provided herein. The forms of VX-950 and the solid dispersions according to this invention may be further processed for preparing a pharmaceutical composition for administering to a patient. Although a solid dispersion could be considered a pharmaceutical composition, further processing may be needed prior to administration (for example, the solid dispersion may be further formulated into a tablet or a liquid suspension). All such pharmaceutical compositions, dosage forms, and pharmaceutical formulations would be included within this invention (e.g., sustained release or immediate release formulations). The formulations may be prepared using known components according to known methods (see, Handbook of Pharmaceutical Excipients). As would be appreciated, oral formulations are often preferred for pharmaceutical administration.

Accordingly, a pharmaceutical composition comprising VX-950 is provided herein. Such compositions typically contain a pharmaceutically acceptable carrier, diluent, or vehicle. In some embodiments, the VX-950 is in amorphous form. In some embodiments, the VX-950 is in the form of a solid dispersion (e.g., an amorphous solid dispersion). These VX-950 forms and dispersions are preferably prepared as disclosed herein.

In one embodiment, the invention includes a pharmaceutical composition that is a suspension formulation including a solid dispersion suspended in a liquid vehicle. It has also been found that preferred compositions are those that include the addition of at least one polymer (e.g., a cellulosic polymer such as HPMC or HPMCAS), not necessarily as a component of the solid dispersion, but possibly as a physical mixture or in solution with the liquid vehicle.

In some embodiments, the polymer helps to prevent the crystallization of supersaturated VX-950 in solution, for example, when amorphous VX-950 is suspended in the liquid vehicle (e.g., water or other aqueous medium). For example, a polymer can be added to the liquid vehicle (e.g., water) and can help to reduce or prevent VX-950 that has become solubilized into the liquid vehicle from crystallizing out of the liquid vehicle. This stabilization can be beneficial as it can provide for improved consistency in liquid dosing. For example, in some embodiments, a liquid suspension including a polymer prepared at time zero with amorphous VX-950 solid dispersion will maintain amorphous VX-950 and therefore have a higher concentration of solubilized VX-950 in the liquid vehicle (e.g., aqueous media) at 2 hours, 4, hours, 12 hours, or 24 hours later, relative to a liquid dispersion including amorphous VX-950 dispersion, but without a polymer added to the liquid vehicle, at the same time intervals. This improvement in consistency of concentration of solubilized amorphous VX-950 is generally due to the inhibition of crystallization by the polymer of the supersatured solubilized VX-950 out of the liquid vehicle. In some preferred embodiments, the polymer can help to prevent amorphous VX-950 formulated into a liquid suspension from becoming crystalline VX-950 for at least about 2 hours, at least about 4 hours, at least about 8 hours, at least about 12 hours, or at least about 24 hours.

Therefore, in another embodiment the pharmaceutical composition comprises a polymer such as a cellulosic polymer or PVP included within a liquid media. Examples of suitable polymers in liquid dispersion formulations include those described for use with solid dispersions above. HPMCs are known to skilled practitioners as polymers that inhibit crystallization (see, e.g., US 2004/0030151).

In some preferred embodiments, one or more hydroxyproply methylcellulose (HPMC) polymer is present in a liquid vehicle used to suspend a solid dispersion. For example, HPMC E50 is present in the liquid vehicle in an amount less than about 10% by weight, (e.g., about 7% by weight, about 5% by weight, about 3% by weight, about 2% by weight, about 1% by weight, about 0.5% by weight, about 0.25% by weight, about 0.1% by weight, or about 0.05% by weight). In some preferred embodiments, the liquid vehicle includes an HPMC polymer present from about 0.1% to about 5% by weight, for example from about 0.2% to about 3% by weight, preferably from about 0.5% to about 1.5% by weight, e.g., about 1% by weight. In some more preferred embodiments, the liquid vehicle includes HPMCAS, for example less than about 10% HPMCAS by weight (about 7% by weight, about 5% by weight, about 3% by weight, about 2% by weight, about 1% by weight, about 0.5% by weight, about 0.25% by weight, about 0.1% by weight, or about 0.05% by weight). In some preferred embodiments, the liquid vehicle includes an HPMCAS polymer present from about 0.1% to about 5% by weight, for example from about 0.2% to about 3% by weight, preferably from about 0.5% to about 1.5% by weight, e.g., about 1% by weight.

In some embodiments, the liquid vehicle includes a surfactant. Such surfactants are as disclosed with the solid dispersions described herein (e.g., Span 65, Span 25, Tween 20, Capryol 90, Pluronic F108, sodium lauryl sulfate (SLS), and Vitamin E TPGS). The amount of surfactant included in a liquid vehicle is dependent on a variety of factors, including the chemical nature of the surfactant. A surfactant is generally present in an amount from about 0% to about 20% by weight (e.g., about 14% by weight, about 13% by weight, about 12% by weight, about 11% by weight, about 10% by weight, about 9% by weight, about 8% by weight, about 7% by weight, about 6% by weight, about 5% by weight, about 4% by weight, about 3% by weight, about 2% by weight, about 1% by weight, or lower). In some preferred embodiments, the surfactant is simethicone (preferably in a an amount of about 0.002% by weight), SLS (e.g., from about 0.25% to about 5% by weight, preferably about 1% by weight), or Vitamin E TPGS (e.g., from about 0.1% to about 20% by weight, preferably about 5% to about 10% by weight). Simethicone is primarily added to reduce foaming.

The compositions and processes of this invention may optionally include one or more excipients (see U.S. Pat. No. 6,720,003, US 2004/0030151, and/or WO 99/02542)). An excipient is a substance used as a carrier or vehicle in a dosage form, or added to a pharmaceutical composition, to improve handling, storage, or preparation of a dosage form. Excipients include, but are not limited to, diluents, disintegrants, adhesives, wetting agents, lubricants, glidants, crystallization inhibitors, surface modifying agents, agents to mask or counteract a disagreeable taste or odor, flavors, dyes, fragrances, fillers, binders, stabilizers and substances to improve the appearance of a composition.

Processes for preparing a formulation comprising an amorphous form of VX-950, or a dispersion or composition thereof, into a dosage form suitable to administration to a mammal are also included herein. Preferably, the formulation comprises a solid dispersion prepared as described herein.

Accordingly, another embodiment of this invention provides a composition comprising VX-950, or a pharmaceutically acceptable salt thereof. According to a preferred embodiment, VX-950 is present in an amount effective to decrease the viral load in a sample or in a patient (e.g., decrease the plasma level of the virus at least about 3 log, at least about 4 log, or at least about 5 log), and a pharmaceutically acceptable carrier. Alternatively, a composition of this invention comprises another additional agent as described herein (e.g., a CYP inhibitor). Each component may be present in individual compositions, combination compositions, or in a single composition.

As used herein the term comprising is intended to be open-ended, thus indicating the potential inclusion of other agents in addition to the specified agents.

As used herein, the compounds of this invention, including VX-950, are defined to include pharmaceutically acceptable derivatives or prodrugs thereof. A “pharmaceutically acceptable derivative or prodrug” means any pharmaceutically acceptable salt, ester, salt of an ester, or other derivative of a compound of this invention (for example an imidate ester of an amide), which, upon administration to a recipient, is capable of providing (directly or indirectly) a compound of this invention. Particularly favored derivatives and prodrugs are those that increase the bioavailability of the compounds of this invention when such compounds are administered to a mammal (e.g., by allowing an orally administered compound to be more readily absorbed into the blood) or which enhance delivery of the parent compound to a biological compartment (e.g., the liver, brain or lymphatic system) relative to the parent species. Preferred prodrugs include derivatives where a group which enhances aqueous solubility or active transport through the gut membrane is appended to the structure of formulae described herein.

The VX-950 utilized in the compositions and methods of this invention may also be modified by appending appropriate functionalities to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion.

Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose based substances, polyethylene glycol, sodium carboxymethylcellulose, polyacrylates, waxes, polyethylene polyoxypropylene block polymers, polyethylene glycol and wool fat.

The pharmaceutical compositions of this invention may be orally administered in any orally acceptable dosage form including, but not limited to, capsules, tablets, pills, powders, granules, aqueous suspensions or solutions. In the case of tablets for oral use, carriers that are commonly used include lactose, microcrystalline cellulose, mannitol, dicalcium phosphate, calcium carbonate and corn starch. Lubricating agents, such as magnesium stearate, sodium stearyl fumerate, or stearic acid, are also typically added. Other ingredients may include disintegrants, such as crosscarmellose sodium or sodium starch glycolate, flow aids such as colloidal silica, and surfactants, such as SLS and Vitamin E, may be included. For oral administration in a capsule form, useful diluents include lactose, microcrystalline cellulose, mannitol, dicalcium phosphate, calcium carbonate and dried cornstarch. Similar to the tablet formulations described above, capsule formulations may also contain lubricants, disintegrants, surfactants, or flow aids. In some embodiments a tablet is coated with a film. When aqueous suspensions are required for oral use, the active ingredient is combined with emulsifying and suspending agents. If desired, certain sweetening, flavoring or coloring agents may also be added. Acceptable liquid dosage forms include emulsions, solutions, suspensions, syrups, and elixirs.

According to a preferred embodiment, the compositions of this invention are formulated for pharmaceutical administration to a mammal, preferably a human being. Although the forms of VX-950 and the dispersions provided herein are preferably formulated for oral administration, other formulations could be obtained.

Other pharmaceutical compositions of the present invention (as well as compositions for use in methods, combinations, kits, and packs of this inventions) may be administered orally, parenterally, sublingually, by inhalation spray, topically, rectally, nasally, buccally, vaginally or via an implanted reservoir. The term “parenteral” as used herein includes subcutaneous, intravenous, intramuscular, intra articular, intra synovial, intrasternal, intrathecal, intrahepatic, intralesional and intracranial injection or infusion techniques. Preferably, the compositions are administered orally or intravenously.

Dosage levels of from about 0.01 to about 100 mg/kg body weight per day, preferably from about 10 to about 100 mg/kg body weight per day of VX-950 are useful for the prevention and treatment of HCV mediated disease. In some embodiments, dosage levels are from about 0.4 to about 10 g/day, for example from about 1 to about 4 g/day, preferably from about 2 to about 3.5 g/day per person (based on the average size of a person calculated at about 70 kg) are included. Typically, the pharmaceutical compositions of, and according to, this invention will be administered from about 1 to about 5 times per day, preferably from about 1 to about 3 times per day, or alternatively, as a continuous infusion. In some embodiments, VX-950 is administered using a controlled release formulation. In some embodiments, this can help to provide relatively stable blood levels of VX-950.

Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Preferably, such preparations contain from about 20% to about 80% active compound.

When the compositions or methods of this invention involve a combination of VX-950 and one or more additional therapeutic or prophylactic agents, both the compound and the additional agent should be present at dosage levels of between about 10 to 100%, and more preferably between about 10 to 80% of the dosage normally administered in a monotherapy regimen.

Upon improvement of a patient's condition, a maintenance dose of a compound, composition or combination of this invention may be administered, if necessary. Subsequently, the dosage or frequency of administration, or both, may be reduced e.g., to about ½ or ¼ or less of the dosage or frequency of administration, as a function of the symptoms, to a level at which the improved condition is retained when the symptoms have been alleviated to the desired level, treatment should cease. Patients may, however, require intermittent treatment on a long-term basis upon any recurrence of disease symptoms.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of active ingredients will also depend upon the particular described compound and the presence or absence and the nature of the additional anti-viral agent in the composition.

The invention also provides pharmaceutical packs and kits comprising amorphous VX-950, a solid dispersion, or a pharmaceutical composition according to any of the embodiments herein.

The invention further provides methods for treating or preventing Hepatitis C virus infection in a patient comprising administering to the patient a pharmaceutical composition. The pharmaceutical composition comprises any form of VX-950, any solid dispersion, or any composition according to this invention.

According to another embodiment, the invention provides a method for treating a patient infected with a virus, e.g., an HCV, characterized by a virally encoded NS3/4A serine protease that is necessary for the life cycle of the virus by administering to said patient any form of VX-950, any solid dispersion, or a composition according of this invention. Preferably, methods of this invention are used to treat a patient suffering from a HCV infection. Such treatment may completely eradicate the viral infection or reduce the severity thereof. More preferably, the patient is a human being.

In yet another embodiment the present invention provides a method of pre-treating a biological substance intended for administration to a patient comprising the step of contacting said biological substance with a pharmaceutically acceptable composition comprising a compound of this invention. Such biological substances include, but are not limited to, blood and components thereof such as plasma, platelets, subpopulations of blood cells and the like; organs such as kidney, liver, heart, lung, etc; sperm and ova; bone marrow and components thereof, and other fluids to be infused into a patient such as saline, dextrose, etc. In some embodiments, VX-950 can be placed on or in a device which is inserted into a patient.

Pharmaceutical compositions may also be prescribed to the patient in “patient packs” containing more than one dose, and preferably the whole course of treatment, in a single package, (e.g., a blister pack). Patient packs have an advantage over traditional prescriptions, where a pharmacist divides a patients supply of a pharmaceutical from a bulk supply, in that the patient always has access to the package insert contained in the patient pack, normally missing in traditional prescriptions. The inclusion of a package insert has been shown to improve patient compliance with the physician's instructions. Preferably the drug is in an oral dosage form.

It will be understood that the administration of the combination of the invention by means of a single patient pack, or patient packs of each formulation, containing within a package insert instructing the patient to the correct use of the invention is a desirable additional feature of this invention.

According to a further aspect of the invention is a pack comprising at least any form of VX-950, any solid dispersion, or any composition according to this invention and an information insert containing directions on the use of the combination of the invention. In an alternative embodiment of this invention, the pharmaceutical pack further comprises one or more of additional agents as described herein. The additional agent or agents may be provided in the same pack or in separate packs.

Another aspect of this involves a packaged kit for inhibiting HCV, or for a patient to use in the treatment of HCV infection or in the prevention of HCV infection, comprising: a single or a plurality of pharmaceutical formulation of each pharmaceutical component; a container housing the pharmaceutical formulation(s) during storage and prior to administration; and instructions for carrying out drug administration in a manner effective to treat or prevent HCV infection. Preferably the drug is in an oral dosage form.

Accordingly, this invention provides kits for the simultaneous or sequential administration of VX-950 (and optionally an additional agent) or derivatives thereof are prepared in a conventional manner. Typically, such a kit will comprise, e.g., a composition of each inhibitor and optionally the additional agent(s) in a pharmaceutically acceptable carrier (and in one or in a plurality of pharmaceutical formulations) and written instructions for the simultaneous or sequential administration. Preferably the drug is in an oral dosage form.

In another embodiment, a packaged kit is provided that contains one or more dosage forms (preferably an oral dosage form) for self administration; a container means, preferably sealed, for housing the dosage forms during storage and prior to use; and instructions for a patient to carry out drug administration. The instructions will typically be written instructions on a package insert, a label, and/or on other components of the kit, and the dosage form or forms are as described herein. Each dosage form may be individually housed, as in a sheet of a metal foil-plastic laminate with each dosage form isolated from the others in individual cells or bubbles, or the dosage forms may be housed in a single container, as in a plastic bottle or a vial. The present kits will also typically include means for packaging the individual kit components, i.e., the dosage forms, the container means, and the written instructions for use. Such packaging means may take the form of a cardboard or paper box, a plastic or foil pouch, etc.

Embodiments of this invention may also involve additional agents. Therefore, a method of this invention may involve steps as administering such additional agents.

Combination Therapy

Methods of this invention may also involve administration of another component comprising an additional agent selected from an immunomodulatory agent; an antiviral agent; an inhibitor of HCV protease; an inhibitor of another target in the HCV life cycle; an inhibitor of internal ribosome entry, a broad-spectrum viral inhibitor; another cytochrome P-450 inhibitor; or combinations thereof.

Accordingly, in another embodiment, this invention provides a method comprising administering any form of VX-950, any solid dispersion, or any composition according to this invention, a CYP inhibitor, and another anti-viral agent, preferably an anti-HCV agent. Such anti-viral agents include, but are not limited to, immunomodulatory agents, such as α-, β-, and γ-interferons, pegylated derivatized interferon-α compounds, and thymosin; other anti-viral agents, such as ribavirin, amantadine, and telbivudine; other inhibitors of hepatitis C proteases (NS2-NS3 inhibitors and NS3/NS4A inhibitors); inhibitors of other targets in the HCV life cycle, including helicase, polymerase, and metalloprotease inhibitors; inhibitors of internal ribosome entry; broad-spectrum viral inhibitors, such as IMPDH inhibitors (e.g., compounds of U.S. Pat. Nos. 5,807,876, 6,498,178, 6,344,465, 6,054,472, WO 97/40028, WO 98/40381, WO 00/56331, and mycophenolic acid and derivatives thereof, and including, but not limited to VX-497, VX-148, and/or VX-944); or combinations of any of the above.

Each agent may be formulated in separate dosage forms. Alternatively, to decrease the number of dosage forms administered to a patient, each agent may be formulated together in any combination. For example, the VX-950 may be formulated in one dosage form and any additional agents may be formulated together or in another dosage form. VX-950 can be dosed, for example, before, after or during the dosage of the additional agent.

A method according to this invention may also comprise the step of administering a cytochrome P450 monooxygenase inhibitor. CYP inhibitors may be useful in increasing liver concentrations and/or increasing blood levels of compounds (e.g., VX-950) that are inhibited by CYP.

The advantages of improving the pharmacokinetics of a drug (e.g., by administering a CYP inhibitor) are well accepted in the art. By administering a CYP inhibitor, this invention provides for decreased metabolism of the protease inhibitor, VX-950. The pharmacokinetics of the protease inhibitor are thereby improved. The advantages of improving the pharmacokinetics of a drug are well accepted in the art. Such improvement may lead to increased blood levels of the protease inhibitor. More importantly for HCV therapies, the improvement may lead to increased concentrations of the protease inhibitor in the liver.

In a method of this invention, the amount of CYP inhibitor administered is sufficient to increase the blood levels of the VX-950 as compared to the blood levels of this protease inhibitor in the absence of a CYP inhibitor. Advantageously, in a method of this invention, an even further lower dose of protease inhibitor may be therefore used (relative to administration of a protease inhibitor alone).

Accordingly, another embodiment of this invention provides a method for increasing blood levels or increasing liver concentrations of VX-950 in a patient receiving VX-950 comprising administering to the patient a therapeutically effective amount of VX-950 and a cytochrome P450 monooxygenase inhibitor.

In addition to treating patients infected with Hepatitis C, the methods of this invention may be used to prevent a patient from becoming infected with Hepatitis C. Accordingly, one embodiment of this invention provides a method for preventing a Hepatitis C virus infection in a patient comprising administering to the patient a) any form of VX-950, any solid dispersion, or any composition according to this invention; and b) a cytochrome P450 monooxygenase inhibitor.

As would be realized by skilled practitioners, if a method of this invention is being used to treat a patient prophylactically, and that patient becomes infected with Hepatitis C virus, the method may then treat the infection. Therefore, one embodiment of this invention provides any form of VX-950, any solid dispersion, or any composition according to this invention and a cytochrome P450 monooxygenase inhibitor wherein the combination of inhibitors are in therapeutically effective amounts for treating or preventing a Hepatitis C infection in a patient.

If an embodiment of this invention involves a CYP inhibitor, any CYP inhibitor that improves the pharmacokinetics of VX-950 may be used in a method of this invention. These CYP inhibitors include, but are not limited to, ritonavir (WO 94/14436), ketoconazole, troleandomycin, 4-methyl pyrazole, cyclosporin, clomethiazole, cimetidine, itraconazole, fluconazole, miconazole, fluvoxamine, fluoxetine, nefazodone, sertraline, indinavir, nelfinavir, amprenavir, fosamprenavir, saquinavir, lopinavir, delavirdine, erythromycin, VX-944 and VX-497. Preferred CYP inhibitors include ritonavir, ketoconazole, troleandomycin, 4-methyl pyrazole, cyclosporin, and clomethiazole. For preferred dosage forms of ritonavir, see U.S. Pat. No. 6,037,157, and the documents cited therein: U.S. Pat. No. 5,484,801, U.S. application Ser. No. 08/402,690, and International Applications WO 95/07696 and WO 95/09614).

The structure of VX-944 is provided below.

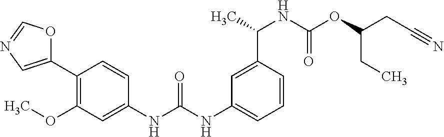

VX-497 is an IMPDH inhibitor. A combination of VX-497, pegylated IFN-α, and ribavirin is currently in clinical development for treating HCV [W. Markland et al., Antimicrobial & Antiviral Chemotherapy, 44, p. 859 (2000); U.S. Pat. No. 6,541,496].

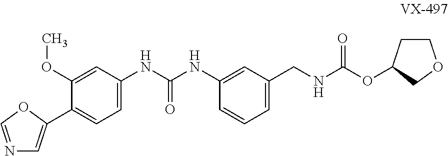 [SMILES: COc1cc(NC(=O)Nc2cccc(CNC(=O)O[C@H]3CCOC3)c2)ccc1-c1cnco1]

Methods for measuring the ability of a compound to inhibit cytochrome P50 monooxygenase activity are known (see U.S. Pat. No. 6,037,157 and Yun, et al. Drug Metabolism & Disposition, vol. 21, pp. 403-407 (1993).

A CYP inhibitor employed in this invention may be an inhibitor of only one isozyme or more than one isozyme. If the CYP inhibitor inhibits more than one isozyme, the inhibitor may nevertheless inhibit one isozyme more selectively than another isozyme. Any such CYP inhibitors may be used in a method of this invention.

In a method of this invention, the CYP inhibitor may be administered together with any form of VX-950, any solid dispersion, or any composition according to this invention in the same dosage form or in separate dosage forms.

If the CYP inhibitor and the other components of the combination are administered in separate dosage forms, each inhibitor may be administered about simultaneously. Alternatively, the CYP inhibitor may be administered in any time period around administration of the combination. That is, the CYP inhibitor may be administered prior to, together with, or following each component of the combination. The time period of administration should be such that the CYP inhibitor affects the metabolism of a component of the combination, preferably, of VX-950. For example, if VX-950 is administered first, the CYP inhibitor should be administered before VX-950 is substantially metabolized and/or excreted (e.g., within the half-life of VX-950).

In order that this invention be more fully understood, the following examples are set forth. These examples are for the purpose of illustration only and are not to be construed as limiting the scope of the invention in any way.

### EXAMPLES

VX-950 may be prepared in general by methods known to those skilled in the art (see, e.g., WO 02/18369). HCV inhibition may be tested in HCV assays according to known methods.

### Example 1

A solid dispersion was prepared comprising the following ingredients (percentage of total weight):

| | |
|---|---|
| VX-950 | 49.5% |
| HPMC 40 cp | 49.5% |
| SLS | 1% |

The composition 1 was prepared by dissolving VX-950, HPMC, and SLS in methanol:methylene chloride (1:1) followed by evaporation of the solvents using rotation evaporation under vacuum. The product was milled to particles with mean particle size of about 200 μm.

### Example 2

A solid dispersion was prepared comprising the following ingredients (percentage of total weight):

| | |
|---|---|
| VX-950 | 49.5% |
| HPC | 49.5% |
| SLS | 1% |

The composition 2 was prepared by dissolving VX-950 and HPC in methylene chloride. SLS was suspended in the solution. The solvent was then evaporated by rotation evaporation under vacuum. The product was milled to particles with mean particle size of about 200 μm.

### Example 3

A solid dispersion was prepared comprising the following ingredients (percentage of total weight):

| | |
|---|---|
| VX-950 | 49.5% |
| PVP K30 | 49.5% |
| SLS | 1% |

The composition 3 was prepared by dissolving VX-950, PVP K30, and suspending SLS in methanol:methylene chloride followed by spray-drying to remove the solvent. The mean particle size of the product is about 150 μm.

### Example 4

A solid dispersion was prepared comprising the following ingredients (percentage of total weight):

| | |
|---|---|
| VX-950 | 49.5% |
| HPMCP | 49.5% |
| SLS | 1% |

The composition 4 was prepared by using a similar procedure as in example 3. The mean particle size of the product is about 150 μm.

Other types of polymers and surfactants were also tested (see the following examples). The ratio of VX-950 and the polymers and the amount of surfactants were also tested in various assays (see the following examples).

### Example 5

Various compositions of VX-950 were tested in a rat pharmacokinetic (PK) assay.

TABLE 1
Rat Pharmacokinetic data
RAT PK

| | | Systemic | Portal |
|---|---|---|---|
| | Dose oral | Plasma | Plasma |
| VX-950 Formulation | (mg/kg) | F (%) | Fa (%) |
| 3 mg/ml Solution in Propylene | 30 | 2.4% | 15.2% |
| Glycol | | | |
| Crystalline Aqueous Suspension | 30 | 1.1% | 4.7% |
| 1% CMC 500 nm Nanosuspension | 30 | 1.7% | 4.0% |
| (crystalline), 3 mg/ml | | | |
| Amorphous Aqueous Suspension, | 30 | 0.4% | 1.4% |
| 3 mg/ml (not a solid dispersion) | | | |

Solid Dispersions

| | | | |
|---|---|---|---|
| 10% VX-950/10% PEG300/10% | 30 | 41.1% | 104.4% |
| SLS/PVP-K30 solvent = | | | |
| EtOH, aqueous dose | | | |
| 10% VX-950/5% SLS/42.5% | 30 | 19.6% | 77.6% |
| PVP-K30/PEG8000, solvent = | | | |
| EtOH, aqueous dose | | | |
| 10% VX-950/10% NMP/10% | 30 | 32.3% | 73.4% |
| SLS/PVP-K30, solvent = | | | |
| EtOH, aqueous dose | | | |
| 10% VX-950/10% PEG300/10% | 30 | 12.7% | 26.6% |
| SLS/PVP-K30, solvent = | | | |
| MeCl/EtOH, aqueous dose | | | |
| 10% VX-950 | 30 | 5.6% | 24.3% |
| solvent = molten | | | |
| PEG-8000, aqueous dose | | | |

### Example 6

Various compositions of VX-950 were tested in a dog pharmacokinetic assay. In this study, the VX-950 compound tested was a 60:40 (+/−5%) mixture of L:D isomers.

TABLE 1
Pharmacokinetic parameters of VX-950
D/L mixture (in dog; 15 mg/kg dose)

| | | Cₘₐₓ | Tₘₐₓ | T_{1/2} | |
|---|---|---|---|---|---|
| Formulation | % F | μg/ml | hr | hr | |
| 20% VRT108720/77% PVP K30/ | 15.12 | 0.89 | 1.33 | 2.25 | Mean |
| 3% SLS | | | | | |
| solid dispersion (EtOH) | 53.85 | 66 | 43 | 31 | CV % |
| 25% VRT108720/72% PVPK30/ | 5.81 | 0.38 | 1.17 | 1.82 | Mean |
| 3% SLS | 20 | 37 | 25 | 34 | CV % |
| 33% VRT108720/64% PVPK30/ | 7.75 | 0.47 | 0.58 | 2.52 | Mean |
| 3% SLS | | | | | |
| Spray-drying | 69.28 | 63 | 65 | 22 | CV % |
| 50% VRT108720/47% PVPK30/ | 18.22 | 1.19 | 1.33 | 2.28 | Mean |
| 3% SLS | | | | | |
| Spray-drying | 38.47 | 41 | 43 | 16 | CV % |
| 20% VRT108720/5% Pluronic | 25.19 | 1.74 | 1.17 | 4.42 | Mean |
| F68/75% Kollidon VA64 | | | | | |
| melt dispersion | 39.79 | 61 | 49 | 22 | CV % |
| 20% VRT108720/5% Labrasol/ | 3.49 | 0.07 | 1.67 | 1.19 | Mean |
| 75% Kollidon VA64 | | | | | |
| melt dispersion | 47.14 | 42 | 35 | 3 | CV % |
| 20% VRT108720/5% Capryol/ | 13.57 | 0.82 | 1 | 1.12 | Mean |
| 75% Kollidon VA64 | | | | | |
| melt dispersion | 77.78 | 41 | 50 | 32 | CV % |
| 20% VRT108720/5% Cremophor/ | 8.91 | 0.63 | 0.75 | 2.34 | Mean |
| 75% Kollidon VA64 | | | | | |
| melt dispersion | 39.85 | 21 | 88 | 40 | CV % |
| 20% VRT108720/5% SLS/75% | 1.55 | 0.13 | 1 | 1.05 | Mean |
| Kollidon VA64 | | | | | |
| melt dispersion | 43.3 | 61 | 50 | 75 | CV % |

### Example 7

The physical stability of various compositions were tested. The results are in Table 2 below.

TABLE 2
Physical stability data
Physical Stability of VX-950 Solid Dispersions

| | | | A = amorphous | | | | |
|---|---|---|---|---|---|---|---|
| | | | C = crystalline | | | | |
| Formulation | | | Blank = not tested | | | | |
| Description | Condition | Lid | 0 | 1 wk | 2 wk | 1 mo | 2 mo |
| amorphous form of | 40 C/75% RH | Closed | A | A | | A | A |
| pure VX-950 (no | 60° C. | Closed | A | A | | A | A |
| polymer) | 25°° C./60% | Closed | A | A | | A | A |
| solvent evaporation, | RH | | | | | | |
| MeCl2 | 40°° C./75% | Open | | | | C | |
| | RH | | | | | | |
| VX-950:PVP K30, | 40° C./75% RH | Closed | A | | | A | A |
| 1:1 | 60° C. | Closed | A | | | A | A |
| 1% SLS | | | | | | | |
| solvent evaporation, | | | | | | | |
| EtOH:MeCl2, 8:2 | | | | | | | |
| | 25°° C./60% | Closed | A | | | A | A |
| | RH | | | | | | |
| VX-950:PVP K30, | 40° C./75% RH | closed | A | | A | A | A |
| 1:1, | 60° C. | closed | A | | A | A | A |
| 1% SLS | 25° C./60% RH | closed | A | | A | A | A |
| spray-dried, | 40° C./75% RH | open | | | | C | |
| MeOH:acetone, 2:1 | | | | | | | |
| VX950:PVP K16, 1:1 | 40° C./75% RH | closed | A | A | | A | A |
| 1% SLS | 60° C. | closed | A | A | | A | A |
| Solvent evaporation, | 25° C./60% RH | closed | A | A | | A | A |
| MeCl2 | 40° C./75% RH | open | | | | A | |

### Example 8

The chiral stability of various compositions were tested. The results are in Table 3 below.

TABLE 3
Chiral stability data
Chiral Stability of 49.5% VX950, 1% SLS, 49.5% Polymer

| | Condition | | % AUC D- |
|---|---|---|---|
| Polymer | (sealed containers) | time | isomer |
| K16 | 25 C./60% RH | 5 mo | 22 |
| K16 | 40 C./75% RH | 5 mo | 28 |
| K30 | 25 C./60% RH | 5 mo | 3 |
| K30 | 40 C./75% RH | 5 mo | 7.5 |

### Example 9

The solubility of various compositions were tested. The results are in Table 4 below.

TABLE 4
Solubility data
Spray-dried Dispersions of VX-950 Absolute
Solubility in Water (measured at 1 hr)

| | | | suspension | Absolute |
|---|---|---|---|---|
| | | Solid load | conc. | solubility, |
| Composition | Solvent | (g/ml) | mg/ml | μg/ml |
| VX950:PVPK30, | MeCl2 | 40% | 50 | 66.87 |
| 1:1, 2% | | | | |
| Pluronic F108 | | | | |
| VX950:HPMC, | MeCl2/t- | 10% | 50 | 399.7 |
| 1:1, 2% SLS | BT, 1:1 | | | |
| VX950:PVPK30, | MeOH/ | 10% | 10 | 41.22 |
| 1:1, 2% | acetone, 2:1 | | | |
| Pluronic F108 | | | | |
| VX-950: PVPK30, | MeCl2 | 10% | 10 | 22.43 |
| 1:1, 2% | | | | |
| Pluronic F108 | | | | |
| VX-950:PVPK30, | MeCl2 | 10% | 10 | 344.2 |
| 1:1, 2% SLS | | | | |
| VX-950:PVPK16, | MeCl2 | 10% | 10 | 277.2 |
| 1:1, 2% SLS | | | | |
| VX-950:PVPK16, | MeCl2 | 10% | 10 | 346.5 |
| 1:2, 2% SLS | | | | |
| VX-950:PVPK16, | MeCl2 | 10% | 10 | 367 |
| 1:1, 1% SLS | | | | |
| VX-950:PVPK30, | MeCl2 | 10% | 10 | 349.5 |
| 1:1, 2% SLS | | | | |

### Example 10

The effect of SLS concentration on the apparent solubility of VX-950 solid dispersions were tested. The results are in Table 5 below.

TABLE 5
Solubility data
Effect of SLS concentration on the apparent
solubility of VX-950 solid dispersions

| VX-950 | % dissolved in water @ |
|---|---|
| (95% L/5% D) | 5 min. |
| No Excipients | 2.7 |
| Only PVP-K30 | 5.6 |
| 0.5% SLS 89.5% PVP | 32.6 |
| 1% SLS 89% PVP | 46.7 |
| 2% SLS 88% PVP | 37.7 |
| 3% SLS 87% PVP | 32.2 |

### Example 11

An oral dosage formulation was prepared as follows. VX-950 and PVP K29/32 were dissolved in methylene chloride, then sodium lauryl sulfate was added and dispersed in the solution to form a homogenous suspension. This suspension was spray-dried using an inlet temperature of 90° C. and an outlet temperature of 56° C., and the product was collected from the cyclone. The spray-dried dispersion was fluid-bed dried at 75° C. for 8 hours.

VX-950 Solid Dispersion

| % (w/w) | Ingredient | |
|---|---|---|
| 49.5 | VX-950 | Spray-dried |
| 49.5 | PVP K29/32 | from a MeCl2 |
| 1 | SLS | solution |

The solid dispersion was suspended in a 1% HPMC, 0.002% simethicone solution using a steel rotary mixer. The resultant suspension is physically and chemically stable at the concentrations of 0.8-50 mg/ml VX-950 for at least 24 hours. The powder is then suspended and dosed within 24 hrs as described in the table below.

Suspension Vehicle

| % | Ingredient | Function |
|---|---|---|
| 1 | Low viscosity hydroxypropyl | Suspending agent |
| | methylcellulose | |
| 0.002 | Simethicone | Anti-foam |
| 99 | Water | diluent |

### Example 12

Dispersions in single dose glass vials mixed with 1% HPMC vehicle were dosed. The solid residue remaining in the vial was 0.8%-4% compared to 28%-56% when dosed in a syringe mixed with water (January 20 dosing below). Dispersions dosed were: VX950/PVPK-30/SLS (tox. lot, refreshed), VX950/HPMCAS/SLS/SDBS (spray dried at ISP starting with crystalline DS containing 5% PVPK-30), VX950/HPMC E15/10% Vit E TPGS, VX950/PVP-VA/10% Vit E TPGS. The results of these studies are provided below.

| | Mean | Mean | |
|---|---|---|---|
| | Cmax | Tmax | Mean |
| Formulation ID (30 mg/Kg dose) | (ng/mL) | (hr) | % F |
| 1:1 VX950: PVPK30, 1% SLS | 981 ± 200 | 0.6 ± 0.3 | 19.6 ± 3.1 |
| (Refreshed Tox.) | | | |
| Niro-49% HPMCAS/1% SLS/1% | 980 ± 200 | 0.9 ± 0.3 | 29.5 ± 4.8 |
| SDBS/49% VX-950 | | | |
| 40.5% PVP-VA/10% ETPGS/ | 1482 ± 400 | 0.5 ± 0.0 | 29.8 ± 9.1 |
| 49.5% VX-950 | | | |
| 40.5% HPMC/10% ETPGS/ | 1890 ± 400 | 0.4 ± 0.1 | 34.7 ± 7.8 |
| 49.5% VX-950 | | | |

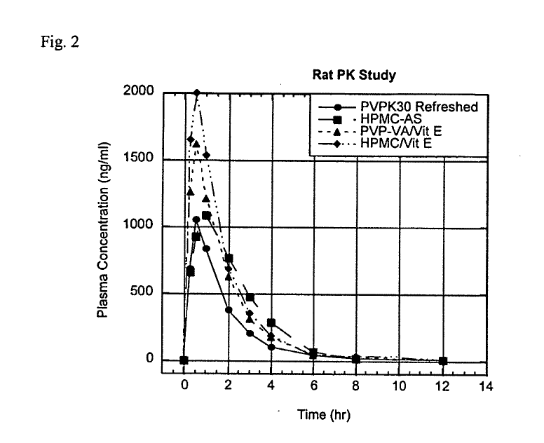

As can be seen in the above table and in FIG. 2, HPMC E-15/10% Vit ETPGS had the highest Cmax and % F (FIG. 2). PVP-VA/10% Vit ETPGS had the second highest Cmax and % F. HPMCAS exhibited a somewhat sustained release profile with a Cmax comparable to PVPK-30 refreshed dispersion and a % F comparable to PVP-VA.

### Example 13

Three formulations were manufactured on the SD Micro spray drier (100 gm). The first 2 formulations had the same ingredients, but varied in acetone levels. The third formulation was a polymer mixture of HPC and HPMC phthalate (2:1). All three formulations contained 1% SLS and 1% SDBS and drug substance that had 5% PVPK-30.

Dissolution of the polymers required homogenization, and all 3 formulations spray-dried very easily. All formulations had detectible residual solvents after manufacture, but both solvents were easily removed with oven drying (60° C.). The addition of acetone appeared to have lowered the initial content of methylene chloride. Residual solvents levels are summarized below

Residual Solvents from Dispersions Manufacture at ISP (100 gm Scale)

| | | | | Residual | |
|---|---|---|---|---|---|
| | | | | Methylene | Residual |
| | | | Drying | Chloride | Acetone |
| Lot# | Formulation | solvent Ratio | Time (hr) | (ppm) | (ppm) |
| 2702-801 | 49% VX950, 49% | 100% | 0 | 10064 | <100 ppm |
| | HPMCAS, 1% | methylene | 1 | 114 | <100 ppm |
| | SLS, 1% SDBS | Chloride | 2 | <100 ppm | <100 ppm |
| | | | 63 | <100 ppm | <100 ppm |
| 2702-802 | 49% VX950, 49% | 30% Acetone/ | 0 | 2889 | 1869 |
| | HPMCAS, 1% | 70% | 1 | <100 ppm | <100 ppm |
| | SLS, 1% SDBS | methylene | 2 | <100 ppm | <100 ppm |
| | | chloride | 63 | <100 ppm | <100 ppm |
| 2702-803 | 49% VX950, 16% | 30% Acetone/ | 0 | 5641 | <100 ppm |
| | HPPh, 33% HPC, | 70% | 1 | <100 ppm | <100 ppm |
| | 1% SLS, 1% | methylene | 2 | <100 ppm | <100 ppm |
| | SDBS | chloride | 63 | <100 ppm | <100 ppm |

### Example 14

A liquid dispersion including HPMCE 50/1% SLS was explored extensively as a suspension in several vehicles at room temperature or refrigerated conditions as follows:

1. 1% HPMC vehicle with varying levels of Vit E TPGS at VX950 concentration of 3 mg/mL.

Solubility and physical stability of the HPMC E50/1% SLS dispersion in suspension containing 0.067%, 1%, 5%, and 10% Vit E TPGS were evaluated using HPLC and XRD according to several procedures to simulate the dosings in the actual tox. studies (b.i.d. dosing, 8-12 hours apart).

Procedure 1: Suspensions made and stored at RT and evaluated at 1, 3, 24, 48 hrs (stirring for 3 hours then stored unstirred until the 24 hrs time point where they're stirred for 15 minutes before sampling).

Procedure 2: Suspensions made at RT but stored at 5° C. after 3 hrs unstirred. At the 24 time point, suspensions were stirred at 5° C. (in ice) before sampling.

Procedure 3: Suspensions made at RT but stored at 5° C. after 3 hrs unstirred. At the 24 time point, suspensions were stirred for 15 minutes at RT (warmed-up) before sampling.

Procedure 4: evaluated only for the 10% Vit E TPGS containing vehicle. Suspensions made and stored at 5° C. and evaluated at 1, 3, 24, 48 hrs (stirring for 3 hours then stored unstirred until the 24 hrs time point where they're stirred for 15 minutes in ice before sampling)

For all the above, kinetic solubility in simulated intestinal fluid at 37° C. was evaluated 1 hr after preparation and after 24 hours of storage under the conditions above.

Results:

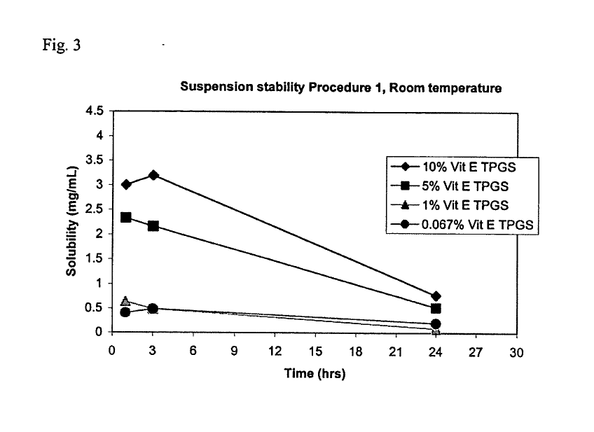

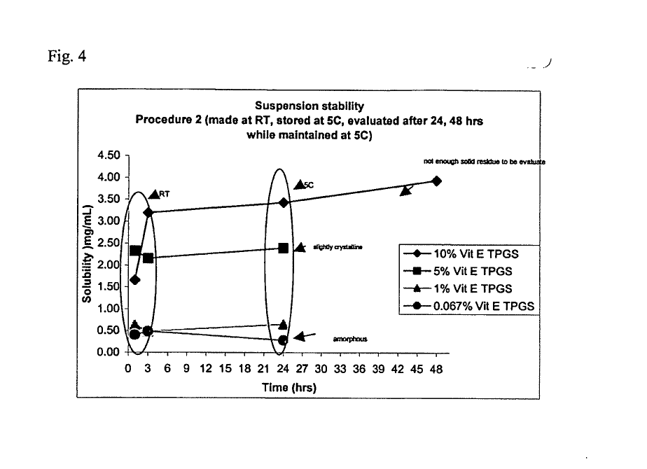

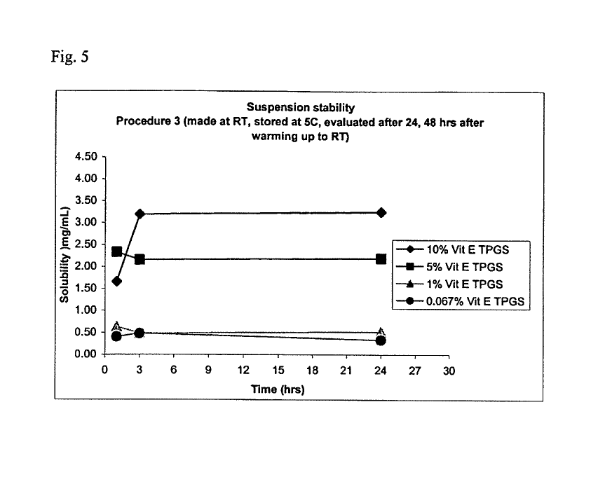

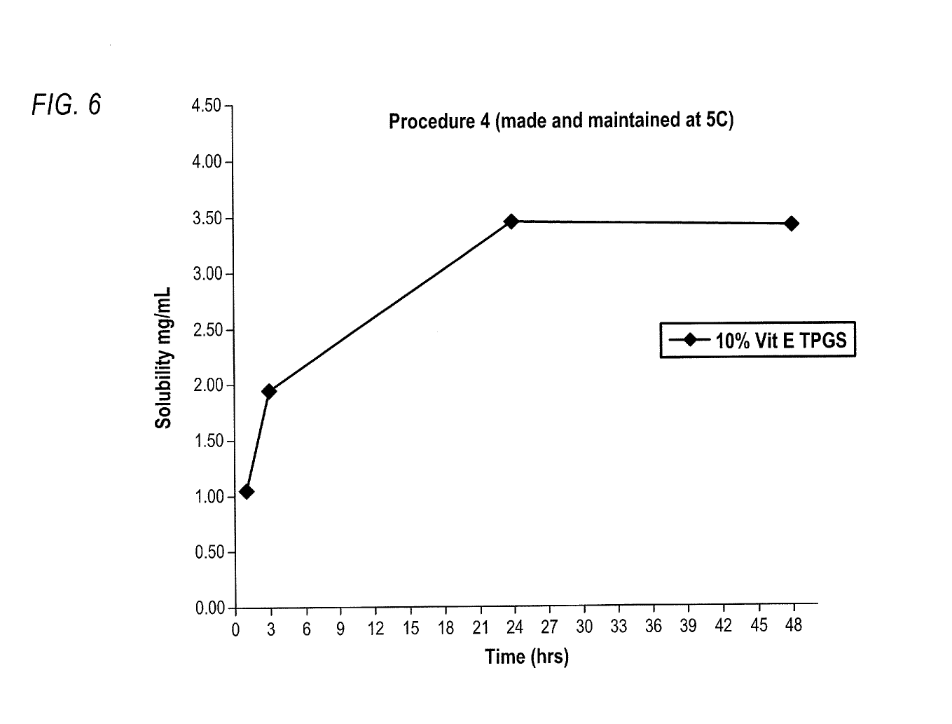

A. Effect of Vit E TPGS level in the 1% HPMC vehicle on suspension solubility is demonstrated in FIGS. 3-6, for the different evaluation/storage procedures.
Procedure 1: Solubility increases as a function of % Vit E TPGS (at 1 and 3 hrs). A significant decrease in solubility is observed after 1 hr for suspensions with the higher levels of Vit E TPGS (10% and 5%) although the actual solubility values remained high 600-700 μg/mL. Collected solid residues dried for 24-48 hrs exhibited some crystallinity. A slight decrease in solubility was observed for the suspension containing 1% Vit E TPGS as well as slight crystallinity. No decrease was observed at the 0.067% Vit E TPGS level and solid residue was amorphous.
Procedure 2: No decrease (change) in solubility was observed at any of the Vit E TPGS levels.
Procedure 3 (warming up): No decrease (change) in solubility was observed at any of the Vit E TPGS levels and the values were the same as in procedure 2
Procedure 4: At 1 and 3 hrs, solubility was lower as compared to procedure 2 (i.e. when made at 5° C. vs at RT), probably due to retarded diffusion/higher viscosity at the lower temperature. No decrease in solubility was observed over 48 hrs and the values were comparable to those obtained in procedure 2 after 24 hrs.

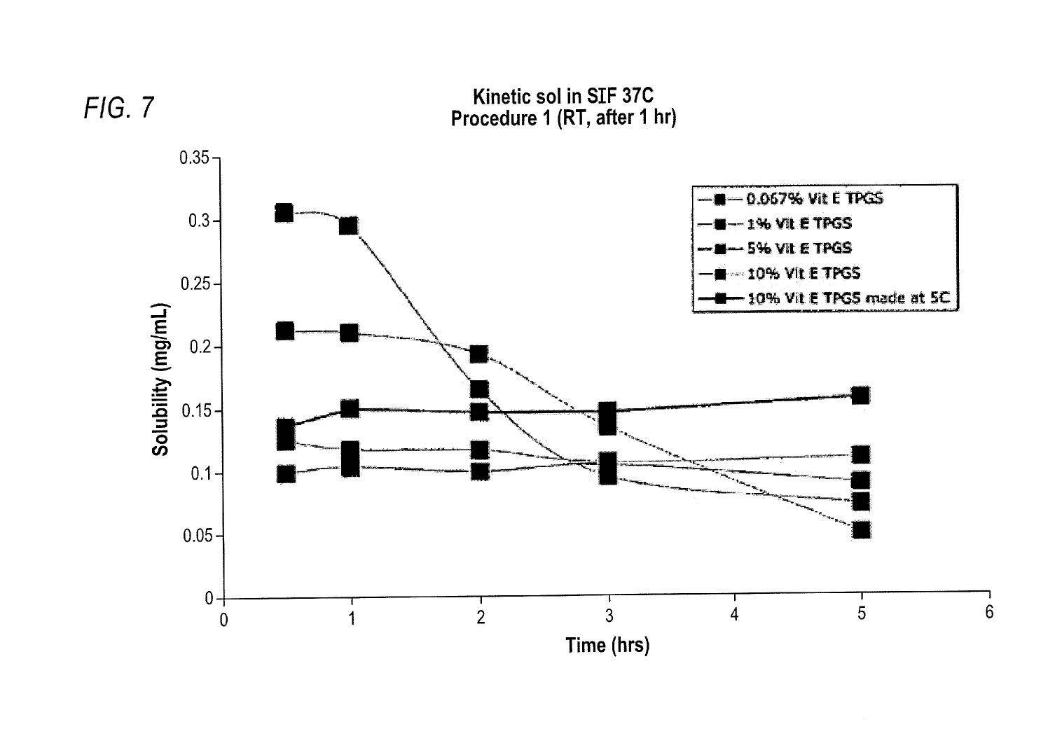

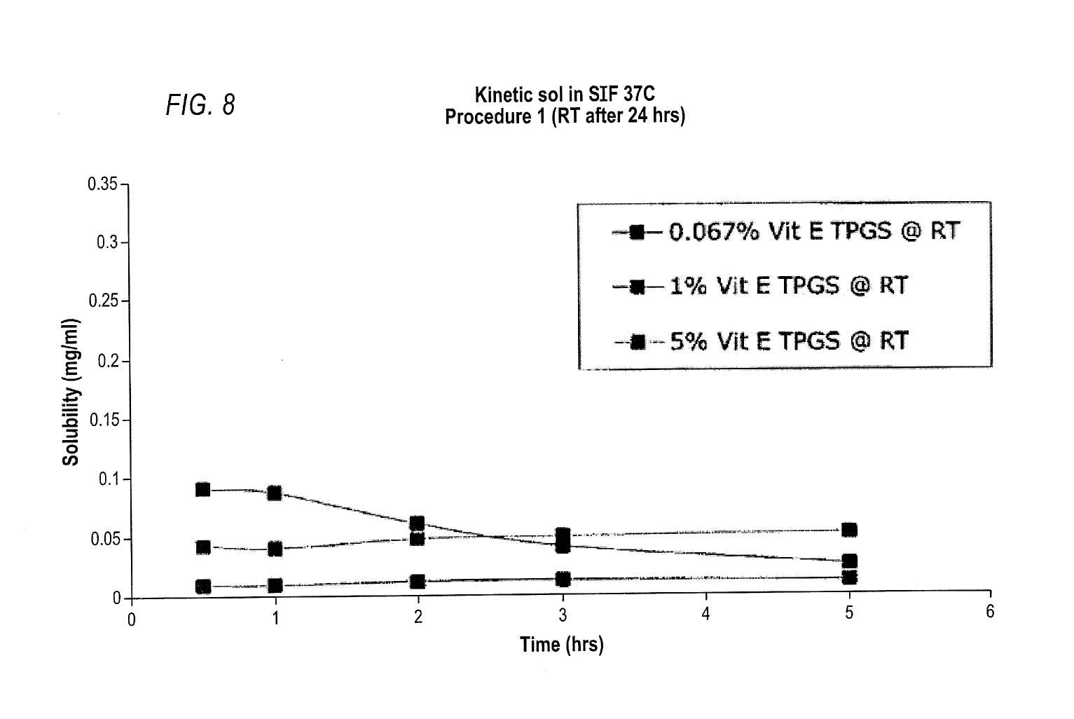

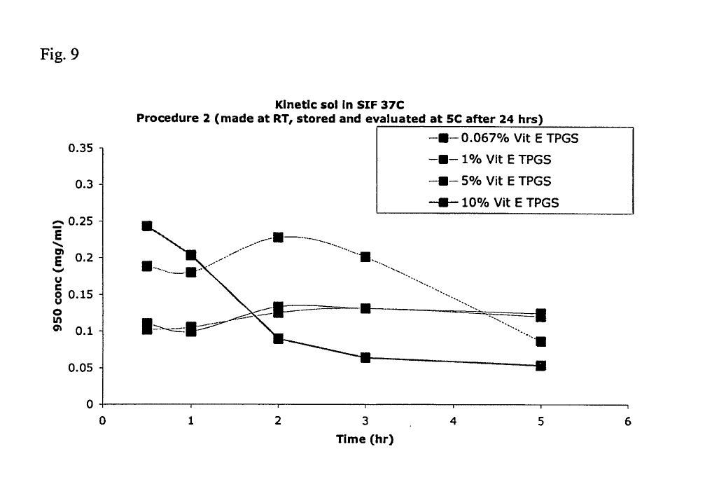

B. Effect of Vit E TPGS level in the 1% HPMC vehicle on kinetic solubility of the suspensions in SIF at 37° C. is demonstrated in FIGS. 7-9, for the different evaluation/storage procedures (5 mL of suspensions at 3 mg/mL VX950 in 50 mL SIF, 37° C.)
Procedure 1, after 1 hr: A significant decrease in solubility is observed at the 10% Vit E TPGS level after 1 hr and a slight decrease is observed at the 5% Vit E TPGS level only after 3 hrs. No decrease was observed at the lower levels (1% and 0.067%) over 5 hrs. In comparison, the suspension containing 10% Vit E TPGS made and stirred on ice (5° C.) for 1 hr shows no decrease in solubility over 5 hrs, however, the actual solubility value is significantly lower than that made at RT. This may explain the reduced % F for the latter in rats.
Procedure 1, after 24 hrs: In comparison to the suspension made and evaluated after 1 hr, the solubility/dissolution is significantly lower for the 1% and 5% Vit E TPGS levels. The 0.067% suspension exhibited initial solubility similar to that observed for the freshly prepared suspension (tested after 1 hr), however a slight decrease in solubility was observed after 2 hrs in SIF, which was not observed for the fresh suspension.
Procedure 2, 24 hrs: similar results as observed for procedure 1 where the suspensions containing lower % Vit E TPGS (0.067% and 1%) showed no decrease in solubility/dissolution after 5 hrs and the absolute values were also the same as those when tested 1 hr after preparation

Conclusions: from the suspension solubility and the kinetic solubility in SIF at 37° C., the suspension containing 0.067% Vit E TPGS exhibited no change in performance (no decrease in suspension solubility over 24 hrs and no decrease in dissolution over 5 hrs for a fresh and a 24 hrs old sample) whether stored at RT or at 5° C. Similar behavior was observed for the suspensions containing 1% and 5% Vit E TPGS only if stored at 5° C. (made at RT).

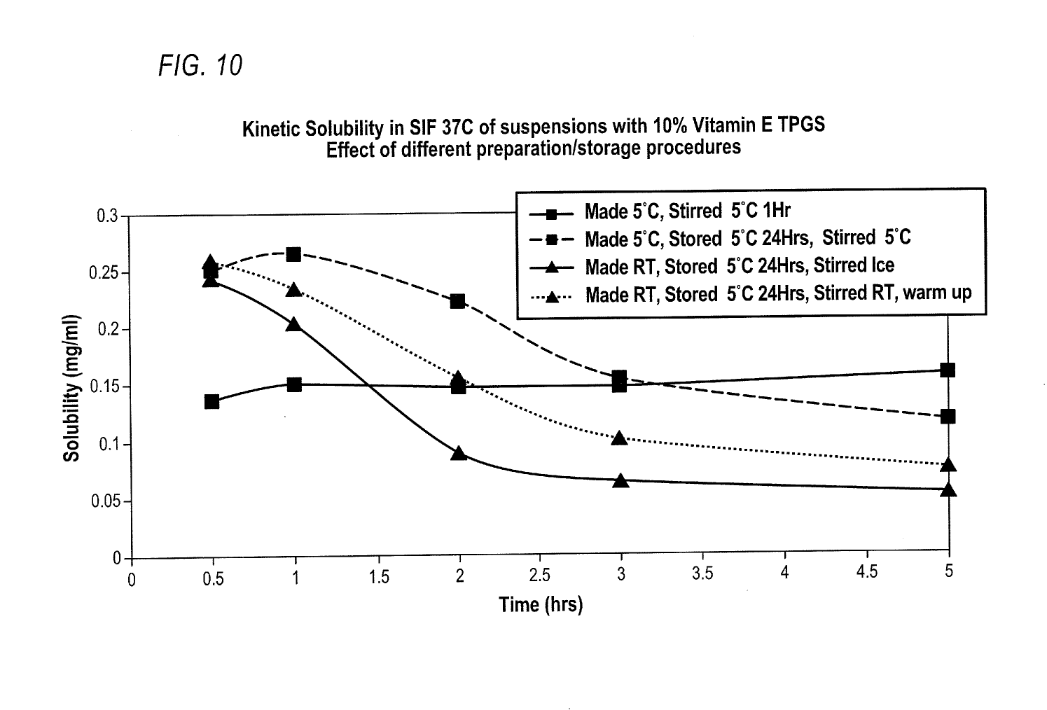

FIG. 10 compares kinetic solubility in SIF (37C) for all 4 evaluation/storage procedures for the suspension containing 10% Vit E TPGS in the vehicle. A gradual decrease in kinetic solubility in SIF at 37° C. was observed over 5 hours for 24 hrs old samples after storage at 5° C. whether warmed to RT or not before evaluation. The suspension made at 5° C. showed lower dissolution/solubility in SIF when evaluated 1 hr after preparation compared to 24 hrs probably due to continued dissolution during storage at 5° C.

All cited document are incorporated herein by reference.

A number of embodiments of the invention have been described. Nevertheless, it will be understood that various modifications may be made without departing from the spirit and scope of the invention. Accordingly, other embodiments are within the scope of the following claims.

What is claimed is:

1. (canceled)

2. (canceled)

3. A spray-dried dispersion comprising VX-950, HPMCAS and about 1% wt/wt sodium lauiyl sulfate.

4. (canceled)

5. (canceled)

6. The spray-dried dispersion of claim 3, wherein less than 40% of the VX-950 is in a crystalline form.

7. The spray-dried dispersion of claim 3, wherein the VX-950 is substantially free of crystalline VX-950.

8. (canceled)

9. (canceled)

10. The spray-dried dispersion of claim 3, wherein the VX-950 contained in the spray-dried dispersion has improved physical or chemical stability relative to VX-950 not in the presence of polymer.

11. The spray-dried dispersion of claim 3, wherein the spray-dried dispersion has a higher glass transition temperature than the glass transition temperature of VX-950 not in a spray-dried dispersion.

12. The spray-dried dispersion of claim 3, wherein the VX-950 contained in the spray-dried dispersion has a relaxation rate that is lower than the relaxation rate of VX-950 not in a spray-dried dispersion.

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. The spray-dried dispersion of claim 3, wherein at least 80% by weight of the VX-950 is in an amorphous form.

30. The spray-dried dispersion of claim 29, wherein substantially all the VX-950 is in an amorphous form.

31. The spray-dried dispersion according to claim 3, wherein the VX-950 is a mixture of the L-isomer and the D-isomer.

32. The spray-dried dispersion according to claim 3, wherein VX-950 is substantially pure L-isomer.

33. (canceled)

34. A pharmaceutical composition comprising the spray-dried dispersion of claim 3.

35. The pharmaceutical composition of claim 34, wherein the VX-950 is substantially free of crystalline VX-950.

36. (canceled)

37. (canceled)

38. The pharmaceutical composition of claim 36, wherein the VX-950 contained in the spray-dried dispersion has improved physical or chemical stability relative to VX-950 not in the presence of a polymer.

39. The pharmaceutical composition of claim 36, wherein the spray-dried dispersion has a higher glass transition temperature than the glass transition temperature of VX-950 not in a spray-dried dispersion.

40. The pharmaceutical composition of claim 36, wherein the VX-950 contained in the spray-dried dispersion has a relaxation rate that is lower than the relaxation rate of VX-950 not in a spray-dried dispersion.

41. (canceled)

42. (canceled)

43. (canceled)

44. (canceled)

45. (canceled)

46. A pharmaceutical composition comprising a mixture of:
VX-950, wherein said VX-950 comprises about 30-75% wt/wt of the pharmaceutical composition,
HPMCAS, wherein
said HPMCAS comprises about 30-75% wt/wt of the pharmaceutical composition, and
sodium lauryl sulfate, wherein said sodium lauryl sulfate comprises about 0.5-2% wt/wt of the pharmaceutical composition,
wherein the mixtures is combined with acetone and methylene chloride and spray-dried to form a solid.

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. A process for preparing VX-950 comprising combining VX-950 and HPMCAS with a suitable solvent and spray-drying the mixture to provide the spray-dried dispersion of claim 3.

63. (canceled)

64. (canceled)

65. (canceled)

66. (canceled)

67. (canceled)

68. (canceled)

69. The process according to claim 62, wherein the solvent comprises methylene chloride.

70. The process of claim 62, wherein the solvent comprises acetone.

71. The process of claim 62, wherein the solvent comprises from about 0% to about 30% acetone and from about 70% to about 100% methylene chloride.

72. The process of claim 62, wherein the solvent comprises from about 0% to about 40% acetone and from about 60% to about 100% methylene chloride.

73. A solid dispersion prepared according to the process of claim 62.

74. (canceled)

75. (canceled)

76. (canceled)

77. (canceled)

78. The solid dispersion of claim 3, wherein the solid dispersion comprises about 49.5% wt/wt VX-950, about 49.5% wt/wt HPMCAS and about 1% wt/wt sodium lauryl sulfate, wherein the solid dispersion is obtained by spray drying.

79. The pharmaceutical composition of claim 46, wherein the pharmaceutical composition comprises:
about 49.5% wt/wt VX-950;
about 49.5% wt/wt HPMCAS; and
about 1% wt/wt sodium lauryl sulfate.
